SEQUENCE LISTING (24 sequences)

SEQ ID NO: 1 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 3 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 4 - protein, 5 aa, synthetic construct
TYAMN

SEQ ID NO: 5 - protein, 19 aa, synthetic construct
RIRSKYNNYATYYADSVKG

SEQ ID NO: 6 - protein, 14 aa, synthetic construct
HGNFGNSYVSWFAY

SEQ ID NO: 7 - protein, 14 aa, synthetic construct
GSSTGAVTTSNYAN

SEQ ID NO: 8 - protein, 7 aa, synthetic construct
GTNKRAP

SEQ ID NO: 9 - protein, 9 aa, synthetic construct
ALWYSNLWV

SEQ ID NO: 10 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 11 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 12 - protein, 5 aa, synthetic construct
YSWIN

SEQ ID NO: 13 - protein, 17 aa, synthetic construct
RIFPGDGDTDYNGKFKG

SEQ ID NO: 14 - protein, 10 aa, synthetic construct
NVFDGYWLVY

SEQ ID NO: 15 - protein, 16 aa, synthetic construct
RSSKSLLHSNGITYLY

SEQ ID NO: 16 - protein, 7 aa, synthetic construct
QMSNLVS

SEQ ID NO: 17 - protein, 9 aa, synthetic construct
AQNLELPYT

SEQ ID NO: 18 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 19 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 20 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 22 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 672 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 24 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

(19) United States
(12) Patent Application Publication
(10) Pub. No.: US 2023/0416412 A1
(43) Pub. Date: Dec. 28, 2023

(54) PREVENTION OR MITIGATION OF T-CELL ENGAGING AGENT-RELATED ADVERSE EFFECTS

(71) Applicant: Hoffmann-La Roche Inc., Little Falls, NJ (US)

(72) Inventors: Gabrielle LECLERCQ-COHEN, Zürich (CH); Johannes SAM, Baden (CH); Nathalie STEINHOFF, Zürich (CH); Pablo UMAÑA, Wollerau (CH)

(73) Assignee: Hoffmann-La Roche Inc., Little Falls, NJ (US)

(21) Appl. No.: 18/324,863

(22) Filed: May 26, 2023

(30) Foreign Application Priority Data
May 31, 2022 (EP) 22176323.8

(51) Int. Cl. C07K 16/46; A61P 43/00

(52) U.S. Cl. CPC C07K 16/468; A61P 43/00; C07K 2317/31; C07K 2317/565; C07K 2317/55

(57) ABSTRACT

The present invention relates to the prevention or mitigation of adverse effects related to T cell engaging agents, such as cytokine release syndrome. Specifically, the invention relates to the prevention or mitigation of such side effects using an inhibitor of NACHT, LRR and PYD domains-containing protein 3 (NLRP3).

### CROSS REFERENCE TO RELATED APPLICATION

This application claims priority under 35 U.S.C. 119(b) to European Patent Application No. 22176323.8, filed May 31, 2022, which is incorporated herein by reference in its entirety.

### SEQUENCE LISTING

This application contains a Sequence Listing which has been submitted electronically in XML format and is hereby incorporated by reference in its entirety. Said XML copy, created on May 26, 2023, is named P37557-US_Sequence_Listing and is 24,270 bytes in size.

### FIELD OF THE INVENTION

The present invention relates to the prevention or mitigation of adverse effects related to T cell engaging agents, such as cytokine release syndrome. Specifically, the invention relates to the prevention or mitigation of such side effects using an inhibitor of NACHT, LRR and PYD domains-containing protein 3 (NLRP3).

### BACKGROUND

T cell engaging agents such as T cell bispecific antibodies (TCBs) or chimeric antigen receptor (CAR) expressing T cells (CAR-T cells) hold great promise as cancer immunotherapeutics. T-cell bispecific antibodies (TCBs) activate T cells by engaging the CD3ε chain of the T-cell receptor and simultaneously bind to the tumor-associated antigens on target cells. They enable the formation of an immunological synapse where the release of pore-forming cytolytic protein (perforin) and of the cytotoxic granule (granzyme B) induce target cell killing. Several TCBs have been described, including CEA-TCB (cibisatamab; Bacac et al. (2016) Clin Cancer Res 22, 3286-97), CD20-TCB (glofitamab; Bacac et al. (2018) Clin Cancer Res 24, 4785-97), WT1-TCB (Augsberger et al. (2021) Blood 138, 2655-69) and BCMA-TCB (Seckinger et al. (2017) Cancer Cell 31, 396-410). Among them, glofitamab (CD20-TCB) has shown potent activity against Diffuse Large B Cell Lymphoma (DLBCL) and Non-Hodgkin's Lymphoma (NHL) (Hutchings et al. (2021) J Clin Oncol 39, 1959-70).

Despite their promising activity, on-target activation of T cells is associated with an intrinsic risk of cytokine release syndrome (CRS), one of the most common safety liabilities associated with the treatment with T cell engaging therapies (see e.g. Shimabukuro-Vornhagen et al. (2018) J Immunother Cancer 6, 56). CRS is characterized by a cytokine storm resulting from an over-activation of T cells and innate immune cells, causing symptoms including fever, hypotension and respiratory deficiency, and in the worst case, multi-organ failure. The ASTCT consensus classifies CRS into different grades based on clinical symptoms (fever, hypotension and hypoxia). For the management of grade 2 and higher CRS, this consensus recommends patient hospitalization and the use of vasopressors and oxygen flow. High dose glucocorticoids and/or IL-6R blockade can also be utilized to alleviate symptoms (Lee et al. (2019) Biol Blood Marrow Transplant 25, 625-38). In the specific case of CD3 bispecific antibodies, step-up or fractionated dosing schedule are widely used in the clinic to lower the risk of first-infusion cytokine storm that may be observed after flat-dose administration. Target cell pre-depletion approaches may also be applied to reduce the amount of circulating and lymphoid tissue resident CD20-expressing B cells and thus restrain the systemic on-target cytokine release by T cell engaging therapies directed against B cell malignancies: one clinically-relevant example is the pre-treatment with obinutuzumab (Gazyva®) in combination with step-up dosing of glofitamab (CD20-TCB), which lowers the rate and severity of CRS. Anti-IL-6 treatment (e.g. with tocilizumab) and glucocorticoids are also used in the management of CRS. Some patients are refractory to these approaches, however, reinforcing the need to develop novel approaches for CRS mitigation.

Although CRS symptoms are managed in the clinic, these remain a frequent dose-limiting safety liability associated with on-target activity of T cell engagers, putting patient safety at risk. Approaches to mitigate these life-threatening toxicities are greatly needed, in particular mitigation strategies that reduce cytokine release while retaining treatment efficacy.

### DESCRIPTION OF THE INVENTION

The present inventors have found that NLRP3 inhibitors may be used to mitigate CRS by T cell engaging therapies. NLRP3 inhibitors such as MCC950 were found to reduce TCB-induced cytokine release while retaining TCB-mediated T cell activation and target cell killing. The results suggest NLRP3 inhibitors as attractive potential superior alternative or complementation to currently used strategies, such as steroids or IL-6/IL-6R blockade, for the mitigation of CRS associated with T cell engaging therapies.

Accordingly, in a first aspect, the present invention provides a T cell engaging agent for use in the treatment of a disease in an individual, wherein said treatment comprises
(a) the administration of the T cell engaging agent to the individual, and
(b) the administration of an NLRP3 inhibitor to the individual.

The invention further provides the use of a T cell engaging agent in the manufacture of a medicament for the treatment of a disease in an individual, wherein said treatment comprises
(a) the administration of the T cell engaging agent to the individual, and
(b) the administration of an NLRP3 inhibitor to the individual.

The invention also provides a method for treatment of a disease in an individual, wherein said method comprises
(a) the administration of a T cell engaging agent to the individual, and
(b) the administration of an NLRP3 inhibitor to the individual.

According to any of the above aspects, the administration of the NLRP3 inhibitor may be for the prevention or mitigation of an adverse effect related to the administration of the T cell engaging agent.

In another aspect, the invention provides an NLRP3 inhibitor for use in the prevention or mitigation of an adverse effect related to the administration of a T cell engaging agent to an individual.

The invention further provides the use of an NLRP3 inhibitor in the manufacture of a medicament for the prevention or mitigation of an adverse effect related to the administration of a T cell engaging agent.

The invention also provides a method for preventing or mitigating an adverse effect related to the administration of a T cell engaging agent to an individual, comprising the administration of an NLRP3 inhibitor to the individual.

The T cell engaging agent for use, NLRP3 inhibitor for use, uses or methods described above and herein, may incorporate, singly or in combination, any of the features described in the following (unless the context dictates otherwise).

Terms are used herein as generally used in the art, unless otherwise defined herein.

“NLRP3” stands for NACHT, LRR and PYD domains-containing protein 3 (also known as NOD-, LRR- and pyrin domain-containing protein 3, NLR (NOD-like receptor) family pyrin domain containing 3, NOD-like receptor protein 3, or cryopyrin). NLRP3 is a tripartite protein that contains an amino-terminal pyrin domain (PIM), a central NACHT domain (domain present in NAIP, CHIA, HET-E and TP1) and a carboxy-terminal leucine-rich repeat domain (LRR domain). The NACHT domain has ATPase activity that is vital for NLRP3 self-association and function, whereas the LRR domain is thought to induce autoinhibition by folding back onto the NACHT domain. NLRP3 is a component of the innate immune system, and expressed predominantly in macrophages. It functions as an intracellular sensor (pattern-recognition receptor (PRR)) that detects a broad range of pathogens and other damage-associated signals, resulting in the formation and activation of the NLRP3 inflammasome. The core of NLRP3 inflammasomes consists of NLRP3 as the signal sensor component, an adapter (ASC, also known as PYCARD), and an effector pro-inflammatory caspase (caspase 1), Assembly of the NLRP3 inflammasome leads to release of pro-inflammatory cytokines as well as to pyroptotic cell death. For a review see e.g. Swanson et al. (2019) Nat Rev Immunol 19, 477-489. Human NLRP3, including its sequence, is also described in UniProt entry no. Q96P20 (version 213).

NLRP3 inhibitors are compounds, in particular small molecule pharmacological inhibitors, that inhibit the NLRP3 inflammasome. For example, NLRP3 inhibitors may disrupt NLRP3-NLRP3 interactions or NLRP3-ASC interactions, or may be directed at the ATP-binding domain of NLRP3 resulting in blockade of its ATPase activity. For a review see e.g. Zahid et al. (2019) Frontiers Immunol 10, Art 2538.

An exemplary NLRP3 inhibitor is MCC950 (also known as CP-456,773), which is a direct inhibitor of NLRP3, reported to target the NLRP3 ATP-hydrolysis motif (Coll et al. (2019) Nat Chem. Biol 15:556-9).

The chemical structure and CAS number of MCC950 are provided below:

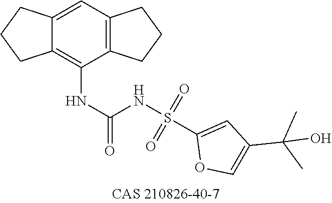 [SMILES: CC(C)(O)c1coc(S(=O)(=O)NC(=O)Nc2c3c(cc4c2CCC4)CCC3)c1]

In some aspects, the NLRP3 inhibitor is a small molecule NLRP3 inhibitor. In some aspects, the NLRP3 inhibitor is a direct inhibitor of NLRP3. In some aspects, the NLRP3 inhibitor blocks die ATPase domain of NLRP3. In some aspects, the NLRP3 inhibitor is MCC950, or a derivative thereof (retaining NLRP3 inhibiting activity). In some aspects, the NLRP3 inhibitor is MCC950 of a salt or ester thereof (e.g. a sodium salt of MCC950).

In some aspects, the NLRP3 inhibitor is selnoflast (Recommended INN: List 87; WHO Drug Information, Vol. 36, No. 1, 2022, page 203), or a salt of ester thereof (e.g. a sodium salt of selnoflast.

The chemical strcture and CAS number of selnoflast are provided below:

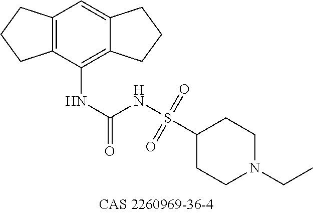 [SMILES: CCN1CCC(S(=O)(=O)NC(=O)Nc2c3c(cc4c2CCC4)CCC3)CC1]

In some aspects, the NLRP3 inhibitor is emlenoflast (Recommended INN: List 87; WHO Drug Information, Vol. 36, No. 1, 2022, page 104), or a salt of ester thereof (e.g. a sodium salt of emlenoflast).

The chemical structure and CAS number of emlenoflast are provided below:

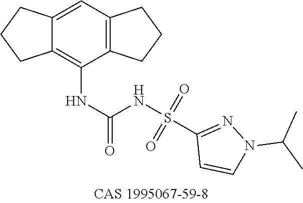 [SMILES: CC(C)n1ccc(S(=O)(=O)NC(=O)Nc2c3c(cc4c2CCC4)CCC3)n1]

In some aspects, the NLRP3 inhibitor is CY-09, or a salt or ester thereof (e.g. a sodium salt of CY-09).

The chemical structure and CAS number of CY-09 are provided below:

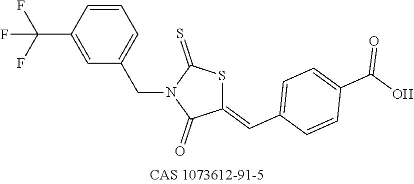 [SMILES: O=C(O)c1ccc(/C=C2\SC(=S)N(Cc3cccc(C(F)(F)F)c3)C2=O)cc1]

Other exemplary NLRP3 inhibitors that might be useful in the present invention include, but are not limited to, IZD334 (also known as Somalix; Inflazome), IZD174 (also known as Inzomelid; Inflazome), DFV890 (also known as IFM-2427; IFM Therapeutics), IFM-632 (IFM Therpeutics), IFM-514 (IFM Therapeutics), JT194 (Jecure Therapeutics), JT349 (Jecure Therapeutics), NT-0167 (NodThera), NT-0796 (NodThera), NT-0249 (NodThera), VENT-01 (Ventus Therapeutics), VTX3232 (Ventyx Biosciences), VTX2735 (Ventyx Biosciences), BT032 (Bacainn Therapeutics), BT132 (Bacainn Therapeutics), OLT1177 (Olatec Therapeutics), ADS-032 (Adiso Therapeutics), ZYIL1 (Zydus Cadila), HY209 (Shaperon), AC-201 (TWi Biotechnology), AC-203 (TWi Biotechnology), and tranilast (also known as Rizaben; Kissei Pharmaceuticals).

In some aspects, the NLRP3 inhibitor is selected from the group consisting of MCC950, selnoflast, emlenoflast, CY-09, IZD334, IZD174, DFV890, IFM-632, IFM-514, JT194, JT349, NT-0167, NT-0796, NT-0249, VENT-01, VTX3232, VTX2735, BT032, BT132, OLT1177, ADS-032, ZYIL1, HY209, AC-201, AC-203, and tranilast. In some aspects, the NLRP3 inhibitor is selected from the group consisting of MCC950, selnoflast, emlenoflast, CY-09, IZD334, and IZD174. In some aspects, the NLRP3 inhibitor is selected from the group consisting of MCC950, selnoflast, emlenoflast.

In some aspects, (administration of) the NLRP3 inhibitor causes inhibition of an activity of the T cell engaging agent. In some aspects, (administration of) the NLRP3 inhibitor does not cause inhibition of another activity of the T cell engaging agent. In some aspects, (administration of) the NLRP3 inhibitor causes inhibition of a first activity of the T cell engaging agent but does not cause inhibition of a second activity of the T cell engaging agent. In some of these aspects, said inhibition is a complete inhibition. In some of these aspects, said inhibition is a partial inhibition. In some aspects, (administration of) the NLRP3 inhibitor causes partial inhibition of a first activity of the T cell engaging agent but does not cause inhibition of a second activity of the T cell engaging agent.

In some aspects, (administration of) the NLRP3 inhibitor causes inhibition of a first activity of the T cell engaging agent and inhibition of a second activity of the T cell engaging agent, wherein said inhibition of the first activity is stronger than said inhibition of the second activity. In some aspects, (administration of) the NLRP3 inhibitor causes inhibition of a first activity of the T cell engaging agent and inhibition of a second activity of the T cell engaging agent, wherein said inhibition of the first activity is a complete inhibition and said inhibition of the second activity is a partial inhibition.

“Activity” of a T cell engaging agent refers to responses in an individual's body caused by the T cell engaging agent. Such activity may include cellular response(s) of T cells, particularly CD4+ and/or CD8+ T cells, such as proliferation, differentiation, cytokine secretion, cytotoxic effector molecule release, cytotoxic activity, and expression of activation markers, and/or effects on target cells, particularly target cells (e.g. tumor cells) expressing the target cell antigen of the T cell engaging agent, such as lysis of target cells.

In some aspects, (administration of) the NLRP3 inhibitor causes inhibition of cytokine secretion by immune cells, particularly T cells (induced by the T cell engaging agent). In some aspects, said cytokine is one or more cytokine selected from the group consisting of IL-1β, IL-6, and IL-8. Immune cells may include various immune cell types, such as T cells, macrophages, monocytes, NK cells etc. In some aspects, said T cells are CD8+ T cells or CD4+ cells. In some aspects, said inhibition is a complete inhibition. In some aspects, said inhibition is a partial inhibition.

In some aspects, (administration of) the NLRP3 inhibitor does not cause inhibition of the activation of T cells (induced by the T cell engaging agent). In some aspects, said inhibition is a complete inhibition. In some aspects, (administration of) the NLRP3 inhibitor causes inhibition of the activation of T cells (induced by the T cell engaging agent), wherein said inhibition is a partial inhibition.

“Activation of T cells” or “T cell activation” as used herein refers to one or more cellular response of a T lymphocyte, particularly a CD4+ or CD8+ T cell, selected from: proliferation, differentiation, cytotoxic effector molecule release, cytotoxic activity, and expression of activation markers. Suitable assays to measure T cell activation are known in the art and described herein. In particular aspects, T cell activation is the expression of activation markers, particularly expression of CD25 and/or CD69 (optionally as measured by flow cytometry). In particular aspects, T cell activation is determined by measuring expression of CD25 and/or CD69 on the T cell, e.g. by flow cytometry.

In some aspects, (administration of) the NLRP3 inhibitor does not cause inhibition of the cytotoxic activity of T cells (induced by the T cell engaging agent). In some aspects, said inhibition is a complete inhibition. In some aspects, (administration of) the NLRP3 inhibitor causes inhibition of the cytotoxic activity of T cells (induced by the T cell engaging agent), wherein said inhibition is a partial inhibition.

“Cytotoxic activity” of a T cell refers to the induction of lysis (i.e. killing) of target cells by a T lymphocyte, particularly a CD4+ or CD8+ T cell. Cytotoxic activity typically involves degranulation of the T lymphocyte, associated with the release of cytotoxic effector molecules such as granzyme B and/or perforin from the T lymphocyte.

In some aspects, (administration of) the NLRP3 inhibitor causes inhibition of cytokine secretion by T cells (induced by the T cell engaging agent) but does not cause inhibition of the activation and/or the cytotoxic activity of T cells (induced by the T cell engaging agent). In some of these aspects, said inhibition is a complete inhibition. In some aspects, said inhibition is a partial inhibition. In some aspects, (administration of) the NLRP3 inhibitor causes partial inhibition of cytokine secretion by T cells (induced by the T cell engaging agent) but does not cause inhibition of the activation and/or the cytotoxic activity of T cells (induced by the T cell engaging agent).

In some aspects, (administration of) the NLRP3 inhibitor causes inhibition of cytokine secretion by T cells (induced by the T cell engaging agent) and inhibition of the activation and/or the cytotoxic activity of T cells (induced by the T cell engaging agent), wherein said inhibition of cytokine secretion is stronger than said inhibition of activation and/or cytotoxic activity. In some aspects, (administration of) the NLRP3 inhibitor causes inhibition of cytokine secretion by T cells (induced by the T cell engaging agent) and inhibition of the activation and/or the cytotoxic activity of T cells (induced by the T cell engaging agent), wherein said inhibition of cytokine secretion is a complete inhibition and said inhibition of activation and/or cytotoxic activity is a partial inhibition.

An inhibition herein may be a partial inhibition or a complete inhibition. A complete inhibition is a stronger inhibition than a partial inhibition. A partial inhibition in some aspects is an inhibition by no more than 30%, no more than 40%, no more than 50%, no more than 60%, or no more than 70%. In some aspects, a partial inhibition is an inhibition by no more than 30%. In some aspects, a partial inhibition is an inhibition by no more than 40%. In some aspects, a partial inhibition is an inhibition by no more than 50%. In some aspects, a partial inhibition is an inhibition by no more than 60%. In some aspects, a partial inhibition is an inhibition by no more than 70%. A complete inhibition in some aspects is an inhibition by at least 80%, at least 90%, or 100%. In some aspects, a complete inhibition is an inhibition by at least 80%. In some aspects, a complete inhibition is an inhibition by at least 90%. In some aspects, a complete inhibition is an inhibition by 100%. In some aspects, a partial inhibition is an inhibition by no more than 50%, and a complete inhibition is an inhibition by at least 80%. In some aspects, a complete inhibition is clinically meaningful and/or statistically significant, and/or a partial inhibition is not clinically meaningful and/or statistically significant.

In some aspects, (administration of) the NLRP3 inhibitor causes reduction of the serum level of one of more cytokine in the individual. In some aspects, (administration of) the NLRP3 inhibitor causes reduction of the secretion of one of more cytokine by immune cells, particularly T cells, in the individual. In some aspects, said one or more cytokine is selected from the group consisting of IL-1β, IL-6, and IL-8. Immune cells may include various immune cell types, such as T cells, macrophages, monocytes, NK cells etc.

In some aspects, said reduction is sustained after the NLRP3 inhibitor has not been administered (to the individual) for a given amount of time. In some aspects, said amount of time is about 1 hour, 2 hours, 3 hours, 4 hours, 5 hours, 6 hours, 7 hours, 8 hours, 12 hours, 16 hours, 20 hours, 24 hours, 36 hours, 48 hours, 72 hours, or 96 hours. In some aspects, said reduction is sustained after a subsequent administration of the T cell engaging agent. Particularly, said reduction is sustained even after administration of the NLRP3 inhibitor is stopped/no further administration of the NLRP3 inhibitor is made. Said reduction of the serum level/cytokine secretion is in particular as compared to the serum level/cytokine secretion in an individual (including the same individual) without administration of the NLRP3 inhibitor (i.e. in such case the serum level/cytokine secretion is reduced as compared to the serum level/cytokine secretion without/before administration of the NLRP3 inhibitor). Said reduction of the serum level/cytokine secretion is in particular as compared to the serum level/cytokine secretion in an individual (including the same individual) with administration (in particular first administration) of the T cell engaging agent but without administration of the NLRP3 inhibitor (i.e. in such case the serum level/cytokine secretion is reduced as compared to the serum level/cytokine secretion with/after administration of the T cell engaging agent but without/before administration of the NLRP3 inhibitor). Without said reduction, the serum level/cytokine secretion particularly may be elevated/increased in relation to the (administration of) the T cell engaging agent. In some aspects, said reduction is clinically meaningful and/or statistically significant. In some aspects, said reduction is at least 30%, at least 40%, at least 50%, at least 60%, or at least 70%. In some aspects, said reduction is at least 30%. In some aspects, said reduction is at least 40%. In some aspects, said reduction is at least 50%. In some aspects, said reduction is at least 60%. In some aspects, said reduction is at least 70%.

In some aspects, (administration of) the NLRP3 inhibitor causes inhibition of an adverse effect related to the administration of the T cell engaging agent. In some aspects, (administration of) the NLRP3 inhibitor does not cause inhibition of a desired effect related to the administration of the T cell engaging agent. In some aspects, (administration of) the NLRP3 inhibitor causes inhibition of an adverse effect related to the administration of the T cell engaging agent but does not cause inhibition of a desired effect related to the administration of the T cell engaging agent. In some of these aspects, said inhibition is a complete inhibition. In some of these aspects, said inhibition is a partial inhibition. In some aspects, (administration of) the NLRP3 inhibitor causes partial inhibition of an adverse effect related to the administration of the T cell engaging agent but does not cause inhibition of a desired effect related to the administration of the T cell engaging agent. In some of these aspects, said inhibition is clinically meaningful and/or statistically significant.

In some aspects, (administration of) the NLRP3 inhibitor causes inhibition of an adverse effect related to the administration of the T cell engaging agent and inhibition of a desired effect related to the administration of the T cell engaging agent, wherein said inhibition of the adverse effect is stronger than said inhibition of the desired effect. In some aspects, (administration of) the NLRP3 inhibitor causes inhibition of an adverse effect related to the administration of the T cell engaging agent and inhibition of a desired effect related to the administration of the T cell engaging agent, wherein said inhibition of the adverse effect is a complete inhibition and said inhibition of the beneficial effect is a partial inhibition. In some aspects, (administration of) the NLRP3 inhibitor causes inhibition of an adverse effect related to the administration of the T cell engaging agent and inhibition of a desired effect related to the administration of the T cell engaging agent, wherein said inhibition of the adverse effect is a clinically meaningful and/or statistically significant inhibition and said inhibition of the beneficial effect is not a clinically meaningful and/or statistically significant inhibition.

A “desired effect” is a beneficial and desired effect resulting from medication in the treatment of an individual, herein particularly with a T cell engaging agent, i.e. a therapeutic or prophylactic effect, such as e.g. killing of tumor cells, reduction or retardation of tumor growth, reduction of tumor volume, reduction or prevention of tumor metastasis, increase of progression-free or overall survival, alleviation of disease symptoms, and the like.

An “adverse effect”, which is sometimes also denoted as “side effect” or “adverse event” (especially in clinical studies is a harmful and undesired effect resulting from medication in the treatment of an individual herein particularly with a T cell engaging agent.

According to the invention, the adverse effect is related to the administration of the T cell engaging agent. In some aspects, the adverse effect is related to the first administration of the T cell engaging agent. In some aspects, the adverse effect occurs upon the first administration of the T cell engaging agent. In some aspects, the adverse effect occurs predominantly or only upon the first administration of the T cell engaging agent. In some aspects, the adverse effect occurs within 12 hours, 24 hours. 36 hours. 48 hours, 72 hours or 96 hours of the administration, particularly the first administration, of the T cell engaging agent. In some aspects, in particular wherein only a single administration of the T cell engaging is made (in the course of the treatment with the T cell engaging agent), the adverse effect occurs within 3 days, 4 days, 5 days, 6 days, 7 days, 10 days, 14 days or 21 days of the administration of the T cell engaging agent.

In some aspects, said adverse effect is cytokine release syndrome (CRS).

“Cytokine release syndrome” (abbreviated as “CRS”) refers to an increase in the levels of cytokines, such tumor necrosis factor alpha (TNF-α), interferon gamma (IFN-γ), interleukin-6 (IL-6), interleukin-10 (IL-10), interleukin-1β (IL-1β), interleukin-8 (IL-8) and others, in the blood of a subject during or shortly after (e.g. within 1 day of) administration of a therapeutic agent (e.g. a T cell engaging agent), resulting in adverse symptoms. CRS is an adverse reaction to therapeutic agent and timely related to administration of the therapeutic agent. It typically occurs during or shortly after an administration of the therapeutic agent, i.e. typically within 24 hours after administration (typically infusion), predominantly at the first administration. In some instances, e.g. after the administration of CAR-T cells, CRS can also occur only later, e.g. several days after administration upon expansion of the CAR-T cells. The incidence and severity typically decrease with subsequent administrations. Symptoms may range from symptomatic discomfort to fatal events, and may include fever, chills, dizziness, hypertension, hypotension, hypoxia, dyspnea, restlessness, sweating, flushing, skin rash, tachycardia, tachypnoea, headache, tumour pain, nausea, vomiting and/or organ failure. CRS may be graded according to the Modified Cytokine Release Syndrome Grading System established by Lee et al., Blood (2014) 124: 188-195 or Lee et al., Biol Blood Marrow Transplant (2019) 25(4): 625-638 (each incorporated herein by reference in its entirety). For a review of CRS see e.g. Shimabukuro-Vomhagen et al., Journal for ImmunoTherapy of Cancer (2018) 6:56 (incorporated herein by reference in its entirety).

In some aspects, said adverse effect is fever, hypotension and/or hypoxia.

In some aspects, said adverse effect is an elevated serum level of one of more cytokine. Said elevated serum level is in particular as compared to the serum level in a healthy individual, and/or the serum level in an individual (including the same individual) without administration of the T cell engaging agent (i.e. in such case the serum level is elevated as compared to the serum level without administration of the T cell engaging agent). In some aspects, said one or more cytokine is selected from the group consisting of IL-1β, IL-6, and IL-8.

In some aspects, administration of the NLRP3 inhibitor is upon (clinical) manifestation of the adverse effect (in the individual). Said administration may be, for example, within about 1 hour, 2 hours, 3 hours, 4 hours, 5 hours, 6 hours, 7 hours, 8 hours, 12 hours, 16 hours, 20 hours or 24 hours after manifestation of the adverse effect (i.e. the occurrence clinical symptoms of the side effect, such as fever). In some aspects, administration of the NLRP3 inhibitor is in response to the (clinical) manifestation of the adverse effect (in the individual).

Administration of the NLRP3 inhibitor may be before, concurrent and/or after the administration of the T cell engaging agent. In some aspects, administration of the NLRP3 inhibitor is before the administration of the T cell engaging agent. In some aspects, administration of the NLRP3 inhibitor is concurrent to the administration of the T cell engaging agent. In some aspects, administration of the NLRP3 inhibitor is after the administration of the T cell engaging agent. Where administration of the NLRP3 inhibitor is before or after the administration of the T cell engaging agent, such administration of the NLRP3 inhibitor may be, for example, within about 1 hour, 2 hours, 3 hours, 4 hours, 5 hours, 6 hours, 7 hours, 8 hours, 12 hours, 16 hours, 20 hours or 24 hours before or after, respectively, the administration of the T cell engaging agent. Administration of the NLRP3 inhibitor may be intermittently or continuously. In some aspects, administration of the NLRP3 inhibitor is oral. In some aspects, administration of the NLRP3 inhibitor is parenteral, particularly intravenous.

In some aspects, administration of the NLRP3 inhibitor is at a dose sufficient to cause inhibition of an activity of the T cell engaging agent. In some aspects, administration of the NLRP3 inhibitor is at a dose insufficient to cause inhibition of another activity of the T cell engaging agent. In some aspects, administration of the NLRP3 inhibitor is at a dose sufficient to cause inhibition of a first activity of the T cell engaging agent but insufficient to cause inhibition of a second activity of the T cell engaging agent. In some of these aspects, said inhibition is a complete inhibition. In some of these aspects, said inhibition is a partial inhibition. In some aspects, administration of the NLRP3 inhibitor is at a dose sufficient to cause partial inhibition of a first activity of the T cell engaging agent but insufficient to cause inhibition of a second activity of the T cell engaging agent.

In some aspects, administration of the NLRP3 inhibitor is at a dose sufficient to cause inhibition of cytokine secretion by immune cells, particularly T cells (induced by the T cell engaging agent). In some aspects, said cytokine is one or more cytokine selected from the group consisting of IL-1β, IL-6, and IL-8. Immune cells may include various immune cell types, such as T cells, macrophages, monocytes, NK cells etc. In some aspects, said T cells are CD8+ T cells or CD4+ cells. In some aspects, said inhibition is a complete inhibition. In some aspects, said inhibition is a partial inhibition.

In some aspects, administration of the NLRP3 inhibitor is at a dose insufficient to cause inhibition of the activation of T cells (induced by the T cell engaging agent). In some aspects, said inhibition is a complete inhibition. In some aspects, said inhibition is a partial inhibition.

In some aspects, administration of the NLRP3 inhibitor is at a dose insufficient to cause inhibition of the cytotoxic activity of T cells (induced by the T cell engaging agent). In some aspects, said inhibition is a complete inhibition. In some aspects, said inhibition is a partial inhibition.

In some aspects, administration of the NLRP3 inhibitor is at a dose sufficient to cause inhibition of cytokine secretion by T cells (induced by the T cell engaging agent) but insufficient to cause inhibition of the activation and/or the cytotoxic activity of T cells (induced by the T cell engaging agent). In some of these aspects, said inhibition is a complete inhibition. In some aspects, said inhibition is a partial inhibition. In some aspects, administration of the NLRP3 inhibitor is at a dose sufficient to cause partial inhibition of cytokine secretion by T cells (induced by the T cell engaging agent) but insufficient to cause inhibition of the activation and/or the cytotoxic activity of T cells (induced by the T cell engaging agent).

In some aspects, administration of the NLRP3 inhibitor is at a dose sufficient to cause reduction of the serum level of one of more cytokine in the individual. In some aspects, administration of the NLRP3 inhibitor is at a dose sufficient to cause reduction of the secretion of one of more cytokine by immune cells, particularly T cells, in the individual. In some aspects, said one or more cytokine is selected from the group consisting of IL-1β, IL-6, and IL-8. Immune cells may include various immune cell types, such as T cells, macrophages, monocytes, NK cells etc.

In some aspects, administration of the NLRP3 inhibitor is at a dose sufficient to cause inhibition of an adverse effect related to the administration of the T cell engaging agent. In some aspects, administration of the NLRP3 inhibitor is at a dose insufficient to cause inhibition of a desired effect related to the administration of the T cell engaging agent. In some aspects, administration of the NLRP3 inhibitor is at a dose sufficient to cause inhibition of an adverse effect related to the administration of the T cell engaging agent but insufficient to cause inhibition of a desired effect related to the administration of the T cell engaging agent. In some of these aspects, said inhibition is a complete inhibition. In some of these aspects, said inhibition is a partial inhibition. In some aspects, administration of the NLRP3 inhibitor is at a dose sufficient to cause partial inhibition of an adverse effect related to the administration of the T cell engaging agent but insufficient to cause inhibition of a desired effect related to the administration of the T cell engaging agent. In some of these aspects, said inhibition is clinically meaningful and/or statistically significant.

In some aspects, administration of the NLRP3 inhibitor is at an effective dose.

An “effective amount” or “effective dose” of an agent, e.g. a NLRP3 inhibitor or a T cell engaging agent, refers to an amount effective, at dosages and for periods of time necessary, to achieve the desired therapeutic or prophylactic result.

In some aspects, the administration of the NLRP3 inhibitor is at a dose equaling a dose strength available for the NLRP3 inhibitor. Typically, several dose strengths (i.e. dosage forms such as tablets or capsules with a specific amount of active ingredient) are available for a given NLRP3 inhibitor. Dosing the NLRP3 inhibitor at such (commercially) available dose strengths will be most convenient.

In some aspects, administration of the NLRP3 inhibitor is daily. In some aspects, administration of the NLRP3 inhibitor is once daily. In some aspects, administration of the NLRP3 inhibitor is once daily at a dose as mentioned hereinabove. In some aspects, administration of the NLRP3 inhibitor is for the period of time during which the adverse effect persists (i.e. administration of the NLRP3 inhibitor is from manifestation of the adverse effect until reduction or disappearance of the adverse effect). In some aspects, administration of the NLRP3 inhibitor is stopped after the adverse effect is prevented or mitigated. In some aspects, administration of the NLRP3 inhibitor is stopped after reduction or disappearance of the adverse effect. Said reduction particularly is clinically meaningful and/or statistically significant. In some aspects, administration of the NLRP3 inhibitor is once, twice, three times, four times, five times, six times, seven times, eight times, nine times or ten times, particularly once, twice, three times, four times, five times, six times, seven times, eight times, nine times or ten times in the course of the treatment of the individual with the T cell engaging agent. In some aspects, administration of the NLRP3 inhibitor is for 1 day, 2 days, 3 days, 4 days, 5 days, 6 days, 7 days, 8 days, 9 days or 10 days. In some aspects, administration of the NLRP3 inhibitor is once daily for 1 day, 2 days, 3 days, 4 days, 5 days, 6 days, 7 days, 8 days, 9 days or 10 days. The administration of the NLRP3 inhibitor is generally associated with the administration of the T cell engaging agent. In some aspects, administration of the NLRP3 inhibitor is associated with the first administration of the T cell engaging agent. Said first administration is particularly the first administration of the T cell engaging agent in the course of the treatment of the individual with the T cell engaging agent. Administration of the NLRP3 inhibitor may be prior to, concurrent with and/or subsequent to the first administration of the T cell engagning agent. In some aspects, administration of the NLRP3 inhibitor is concurrent with the first administration of the T cell engaging agent. In some aspects, administration of the NLRP3 inhibitor is prior to the first administration of the T cell engaging agent. In some aspects, administration of the NLRP3 inhibitor is subsequent to the first administration of the T cell engaging agent. In some aspects, administration of the NLRP3 inhibitor is subsequent to the first administration of the T cell engaging agent and prior to a second administration of the T cell engaging agent. Where administration of the NLRP3 inhibitor is prior or subsequent to the (first) administration of the T cell engaging agent, such administration of the NLRP3 inhibitor may be, for example, within about 1 hour, 2 hours, 3 hours, 4 hours, 5 hours, 6 hours, 7 hours, 8 hours, 12 hours, 16 hours, 20 hours, 24 hours, 48 hours or 72 hours before or after, respectively, the administration of the T cell engaging agent.

In some aspects, the administration of the T cell engaging agent is for a longer period of time than the administration of the NLRP3 inhibitor. In some aspects, the administration of the T cell engaging agent continues after the administration of the NLRP3 inhibitor is stopped. In some aspects, the administration of the T cell engaging agent is a single administration or a repeated administration. In the course of the treatment of the individual with the T cell engaging agent, the T cell engaging agent may be administered once or several times. For example, treatment of the individual with the T cell engaging agent may comprise multiple treatment cycles which each comprise one or more administrations of the T cell engaging agent. In some aspects, the administration of the T cell engaging agent comprises a first and a second administration.

For use in the present invention, the T cell engaging agent would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In some aspects, the administration of the T cell engaging agent is at an effective dose. For systemic administration, an effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. Dosage amount and interval may be adjusted individually to provide plasma levels of the T cell engaging agent which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

An effective amount of the T cell engaging agent may be administered for prevention or treatment of disease. The appropriate route of administration and dosage of the T cell engaging agent may be determined based on the type of disease to be treated, the type of the T cell engaging agent, the severity and course of the disease, the clinical condition of the individual, the individual's clinical history and response to the treatment, and the discretion of the attending physician. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The T cell engaging agent and the NLRP3 inhibitor can be administered by any suitable route, and may be administered by the same route of administration or by different routes of administration. In some aspects, the administration of the T cell engaging agent is parenteral, particularly intravenous.

In some aspects, the administration of the T cell engaging agent is the first administration of the T cell engaging agent to the individual, particularly the first administration of the T cell engaging agent in the course of the treatment of the individual with the T cell engaging agent.

In some aspects, (administration of) the T cell engaging agent induces (i.e. causes or increases) the activation of T cells. In some aspects, (administration of) the T cell engaging agent induces cytotoxic activity of T cells. In some aspects, (administration of) the T cell engaging agent induces cytokine secretion by T cells. In some aspects, cytokine is one or more cytokine selected from the group consisting of IL-2, IL-6, IL-1β, IL-8, IFN-γ, IP-10, TNF-α, IL-1Ra, MCP-1 and MIP-1α. In some aspects, cytokine is one or more cytokine selected from the group consisting of IL-1β, IL-6, and IL-8. In some aspects, said T cells are CD8+ T cells or CD4+ cells.

In some aspects, administration of the T cell engaging agent results in activation of T cells, particularly cytotoxic T cells, particularly at the site of the cancer (e.g. within a solid tumor cancer). Said activation may comprise proliferation of T cells, differentiation of T cells, cytokine secretion by T cells, cytotoxic effector molecule release from T cells, cytotoxic activity of T cells, and expression of activation markers by T cells. In some aspects, the administration of the T cell engaging agent results in an increase of T cell, particularly cytotoxic T cell, numbers at the site of the cancer (e.g. within a solid tumor cancer).

By “T cell engaging agent” is meant an immunotherapeutic agent that exerts its effect through the activity of T cells, particularly cytotoxic T cells. Such activity of T cells may include cellular response(s) of T cells, particularly CD4+ and/or CD8+ T cells, such as proliferation, differentiation, expression of activation markers, cytokine secretion, cytotoxic effector molecule release and/or cytotoxic activity. T cell engaging agents as contemplated herein typically comprise an antigen binding moiety that enables their binding to a target cell antigen on a target cell such as a tumor cell. Such T cell engaging agents exert effects on their target cell, such as lysis of the target cell, through the activity of T cells. Exemplary T cell engaging agents include T cell bispecific antibodies, chimeric antigen receptor (CAR) expressing T cells (CAR-T cells), and T cell receptor (TCR)-based approaches such as ImmTACs (“Immune mobilising monoclonal T-cell receptors Against Cancer”; bispecific fusion proteins of an engineered TCR and an antibody fragment, capable of binding to a T cell and a target cell) or TCR-modified T cells featuring engineered T cell receptors capable of binding to a specific antigenic determinant on a target cell (TCR-T cells).

In particular aspects of the present invention, the T cell engaging agent is a T cell bispecific antibody.

In other aspects, the T cell engaging agent is a CAR-T cell. In some aspects, the T cell engaging agent is an ImmTAC. In some aspects, the T cell engaging agent is a TCR-T cell.

In the following, the T cell bispecific antibody that may be used in the present invention is described.

By “T cell bispecific antibody” is meant an antibody that is able to bind, including simultaneously bind, to a T cell (typically via an antigenic determinant expressed on the T cell, such as CD3) and to a target cell (typically via an antigenic determinant expressed on the target cell, such as CD20).

In preferred aspects according to the invention, the T cell bispecific antibody is capable of simultaneous binding to the antigenic determinant on the T cell (i.e. a first antigen such as CD3) and the antigenic determinant on the target cell (i.e. a second antigen such as CD20). In some aspects, the T cell bispecific antibody is capable of crosslinking the T cell and the target cell by simultaneous binding to CD3 and a target cell antigen. In even more preferred aspects, such simultaneous binding results in lysis of the target cell, particularly a target cell antigen (e.g. CD20)-expressing tumor cell. In some aspects, such simultaneous binding results in activation of the T cell. In some aspects, such simultaneous binding results in a cellular response of the T cell, selected from the group of: proliferation, differentiation, cytokine secretion, cytotoxic effector molecule release, cytotoxic activity, and expression of activation markers. In some aspects, binding of the T cell bispecific antibody to CD3 without simultaneous binding to the target cell antigen does not result in T cell activation. In some aspects, the T cell bispecific antibody is capable of re-directing cytotoxic activity of a T cell to a target cell. In preferred aspects, said re-direction is independent of MHC-mediated peptide antigen presentation by the target cell and and/or specificity of the T cell.

The term “bispecific” means that the antibody is able to bind to at least two distinct antigenic determinants. Typically, a bispecific antibody comprises two antigen binding sites, each of which is specific for a different antigenic determinant. In certain aspects, the bispecific antibody is capable of simultaneously binding two antigenic determinants, particularly two antigenic determinants expressed on two distinct cells.

As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

As used herein, the term “antigen binding moiety” refers to a polypeptide molecule that binds, including specifically binds, to an antigenic determinant. In some aspects, an antigen binding moiety is able to direct the entity to which it is attached (e.g. a second antigen binding moiety) to a target site, for example to a specific type of tumor cell bearing the antigenic determinant. In further aspects, an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain aspects, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

By “specific binding” is meant that the binding is selective for the antigen and can be discriminated from unwanted or non-specific interactions. The term “bind” or “binding” herein generally refers to “specific binding”. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In some aspects, the extent of binding of an antigen binding moiety to an unrelated protein is less than about 10% of the binding of the antigen binding moiety to the antigen as measured, e.g., by SPR. In certain aspects, an antigen binding moiety that binds to the antigen, or an antibody comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In some aspects, CD3 is human CD3, particularly the epsilon subunit of human CD3 (CD3c). The amino acid sequence of human CD3ε is shown in UniProt (www.uniprot.org) accession no. P07766 (version 144), or NCBI (www.ncbi.nlm.nih.gov/) RefSeq NP_000724.1. See also SEQ ID NO: 1. The amino acid sequence of cynomolgus [Macaca fascicularis] CD3ε is shown in NCBI GenBank no. BAB71849.1. See also SEQ ID NO: 2.

A “target cell antigen” as used herein refers to an antigenic determinant presented on the surface of a target cell, for example a cell in a tumor such as a cancer cell or a cell of the tumor stroma (in that case a “tumor cell antigen”). Preferably, the target cell antigen is not CD3, and/or is expressed on a different cell than CD3. In some aspects, the target cell antigen is CD20, particularly human CD20.

As used herein, the terms “first”, “second” or “third” with respect to antigen binding moieties etc., are used for convenience of distinguishing when there is more than one of each type of moiety. Use of these terms is not intended to confer a specific order or orientation of the bispecific antibody unless explicitly so stated.

The term “valent” as used herein denotes the presence of a specified number of antigen binding sites in an antibody. As such, the term “monovalent binding to an antigen” denotes the presence of one (and not more than one) antigen binding site specific for the antigen in the antibody.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain aspects, a single-domain antibody is a human single-domain antibody (Domantis, Inc., Waltham, M A; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W. H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1 (L1)-FR2-H2(L2)-FR3-H3 (L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some aspects the modification promoting association comprises an amino acid mutation in the Fc domain, specifically an amino acid substitution. In particular aspects, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “fused” is meant that the components (e.g. a Fab molecule and an Fc domain subunit) are linked by peptide bonds, either directly or via one or more peptide linkers.

In particular aspects, the T cell bispecific antibody binds to CD3 and a target cell antigen. Accordingly, in some aspects, the T cell bispecific antibody comprises an antigen binding moiety that binds to CD3 and an antigen binding moiety that binds to a target cell antigen.

In some aspects, the first and/or the second antigen binding moiety is a Fab molecule. In some aspects, the first antigen binding moiety is a crossover Fab molecule wherein either the variable or the constant regions of the Fab light chain and the Fab heavy chain are exchanged. In such aspects, the second antigen binding moiety preferably is a conventional Fab molecule.

In some aspects wherein the first and the second antigen binding moiety of the T cell bispecific antibody are both Fab molecules, and in one of the antigen binding moieties (particularly the first antigen binding moiety) the variable domains VL and VH of the Fab light chain and the Fab heavy chain are replaced by each other,
i) in the constant domain CL of the first antigen binding moiety the amino acid at position 124 is substituted by a positively charged amino acid (numbering according to Kabat), and wherein in the constant domain CH1 of the first antigen binding moiety the amino acid at position 147 or the amino acid at position 213 is substituted by a negatively charged amino acid (numbering according to Kabat EU index); or
ii) in the constant domain CL of the second antigen binding moiety the amino acid at position 124 is substituted by a positively charged amino acid (numbering according to Kabat), and wherein in the constant domain CH1 of the second antigen binding moiety the amino acid at position 147 or the amino acid at position 213 is substituted by a negatively charged amino acid (numbering according to Kabat EU index).

The T cell bispecific antibody does not comprise both modifications mentioned under i) and ii). The constant domains CL and CH1 of the antigen binding moiety having the VH/VL exchange are not replaced by each other (i.e. remain unexchanged).

In more specific aspects,
i) in the constant domain CL of the first antigen binding moiety the amino acid at position 124 is substituted independently by lysine (K), arginine (R) or histidine (H) (numbering according to Kabat), and in the constant domain CH1 of the first antigen binding moiety the amino acid at position 147 or the amino acid at position 213 is substituted independently by glutamic acid (E), or aspartic acid (D) (numbering according to Kabat EU index); or
ii) in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In some aspects, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In further aspects, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index).

In preferred aspects, in the constant domain CL of the second antigen binding moiety the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In some aspects, in the constant domain CL of the second antigen binding moiety the amino acid at position 124 is substituted by lysine (K) (numbering according to Kabat) and the amino acid at position 123 is substituted by lysine (K) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In some aspects, in the constant domain CL of the second antigen binding moiety the amino acid at position 124 is substituted by lysine (K) (numbering according to Kabat) and the amino acid at position 123 is substituted by arginine (R) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In particular aspects, if amino acid substitutions according to the above aspects are made in the constant domain CL and the constant domain CH1 of the second antigen binding moiety, the constant domain CL of the second antigen binding moiety is of kappa isotype.

In some aspects, the first and the second antigen binding moiety are fused to each other, optionally via a peptide linker.

In some aspects, the first and the second antigen binding moiety are each a Fab molecule and either (i) the second antigen binding moiety is fused at the C-terminus of the Fab heavy chain to the N-terminus of the Fab heavy chain of the first antigen binding moiety, or (ii) the first antigen binding moiety is fused at the C-terminus of the Fab heavy chain to the N-terminus of the Fab heavy chain of the second antigen binding moiety.

In some aspects, the T cell bispecific antibody provides monovalent binding to CD3.

In particular aspects, the T cell bispecific antibody comprises a single antigen binding moiety that binds to CD3, and two antigen binding moieties that bind to the target cell antigen. Thus, in some aspects, the T cell bispecific antibody comprises a third antigen binding moiety, particularly a Fab molecule, more particularly a conventional Fab molecule, that binds to the target antigen. The third antigen binding moiety may incorporate, singly or in combination, all of the features described herein in relation to the second antigen binding moiety (e.g. the CDR sequences, variable region sequences, and/or amino acid substitutions in the constant regions). In some aspects, the third antigen moiety is identical to the first antigen binding moiety (e.g. is also a conventional Fab molecule and comprises the same amino acid sequences).

In particular aspects, the T cell bispecific antibody further comprises an Fc domain composed of a first and a second subunit. In some aspects, the Fc domain is an IgG Fc domain. In particular aspects, the Fc domain is an IgG₁ Fc domain. In other aspects, the Fc domain is an IgG₄ Fc domain. In more specific aspects, the Fc domain is an IgG₄ Fc domain comprising an amino acid substitution at position 5228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In further particular aspects, the Fc domain is a human Fc domain. In particularly preferred aspects, the Fc domain is a human IgG₁ Fc domain. An exemplary sequence of a human IgG₁ Fc region is given in SEQ ID NO: 3.

In some aspects wherein the first, the second and, where present, the third antigen binding moiety are each a Fab molecule, (a) either (i) the second antigen binding moiety is fused at the C-terminus of the Fab heavy chain to the N-terminus of the Fab heavy chain of the first antigen binding moiety and the first antigen binding moiety is fused at the C-terminus of the Fab heavy chain to the N-terminus of the first subunit of the Fc domain, or (ii) the first antigen binding moiety is fused at the C-terminus of the Fab heavy chain to the N-terminus of the Fab heavy chain of the second antigen binding moiety and the second antigen binding moiety is fused at the C-terminus of the Fab heavy chain to the N-terminus of the first subunit of the Fc domain; and (b) the third antigen binding moiety, where present, is fused at the C-terminus of the Fab heavy chain to the N-terminus of the second subunit of the Fc domain.

In some aspects, the T cell bispecific antibody essentially consists of the first, the second and the third antigen binding moiety (particularly Fab molecule), the Fc domain composed of a first and a second subunit, and optionally one or more peptide linkers.

The components of the T cell bispecific antibody may be fused to each other directly or, preferably, via one or more suitable peptide linkers. Where fusion of a Fab molecule is to the N-terminus of a subunit of the Fc domain, it is typically via an immunoglobulin hinge region.

The antigen binding moieties may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ, G₄(SG₄)ₙ or (G₄S)ₙG₅ peptide linkers. “n” is generally an integer from 1 to 10, typically from 2 to 4. In some aspects, said peptide linker has a length of at least 5 amino acids, in some aspects a length of 5 to 100, in further aspects of 10 to 50 amino acids. In some aspects said peptide linker is (G×S)ₙ or (G×S)ₙGₘ with G=glycine, S=serine, and (x=3, n=3, 4, 5 or 6, and m=0, 1, 2 or 3) or (x=4, n=1, 2, 3, 4 or 5 and m=0, 1, 2, 3, 4 or 5), in some aspects x=4 and n=2 or 3, in further aspects x=4 and n=2, in yet further aspects x=4, n=1 and m=5. In some aspects, said peptide linker is (G₄S)₂. In other aspects, said peptide linker is G₄SG₅. Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In particular aspects, the Fc domain comprises a modification promoting the association of the first and the second subunit of the Fc domain. The site of most extensive protein-protein interaction between the two subunits of a human IgG Fc domain is in the CH3 domain. Thus, in some aspects, said modification is in the CH3 domain of the Fc domain.

In specific aspects, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in some aspects, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g. by site-specific mutagenesis, or by peptide synthesis.

In specific such aspects, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In further aspects, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In preferred aspects, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some aspects, the Fc domain comprises one or more amino acid substitution that reduces binding to an Fc receptor and/or effector function.

In particular aspects, the Fc receptor is an Fcγ receptor. In some aspects, the Fc receptor is a human Fc receptor. In some aspects, the Fc receptor is an activating Fc receptor. In specific aspects, the Fc receptor is an activating human Fcγ receptor, more specifically human FcγRIIIa, FcγRI or FcγRIIa, most specifically human FcγRIIIa. In some aspects, the effector function is one or more selected from the group of complement dependent cytotoxicity (CDC), antibody-dependent cell-mediated cytotoxicity (ADCC), antibody-dependent cellular phagocytosis (ADCP), and cytokine secretion. In particular aspects, the effector function is ADCC.

Typically, the same one or more amino acid substitution is present in each of the two subunits of the Fc domain. In some aspects, the one or more amino acid substitution reduces the binding affinity of the Fc domain to an Fc receptor. In some aspects, the one or more amino acid substitution reduces the binding affinity of the Fc domain to an Fc receptor by at least 2-fold, at least 5-fold, or at least 10-fold.

In some aspects, the Fc domain comprises an amino acid substitution at a position selected from the group of E233, L234, L235, N297, P331 and P329 (numberings according to Kabat EU index). In more specific aspects, the Fc domain comprises an amino acid substitution at a position selected from the group of L234, L235 and P329 (numberings according to Kabat EU index). In some aspects, the Fc domain comprises the amino acid substitutions L234A and L235A (numberings according to Kabat EU index). In some such aspects, the Fc domain is an IgG₁ Fc domain, particularly a human IgG₁ Fc domain. In some aspects, the Fc domain comprises an amino acid substitution at position P329. In more specific aspects, the amino acid substitution is P329A or P329G, particularly P329G (numberings according to Kabat EU index). In some aspects, the Fc domain comprises an amino acid substitution at position P329 and a further amino acid substitution at a position selected from E233, L234, L235, N297 and P331 (numberings according to Kabat EU index). In more specific aspects, the further amino acid substitution is E233P, L234A, L235A, L235E, N297A, N297D or P331S. In particular aspects, the Fc domain comprises amino acid substitutions at positions P329, L234 and L235 (numberings according to Kabat EU index). In more particular aspects, the Fc domain comprises the amino acid mutations L234A, L235A and P329G (“P329G LALA”, “PGLALA” or “LALAPG”). Specifically, in preferred aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In some such aspects, the Fc domain is an IgG₁ Fc domain, particularly a human IgG₁ Fc domain.

In some aspects, the target cell antigen of the T cell bispecific antibody is CD20.

“CD20”, also known as “B-lymphocyte antigen B1”, refers to any native CD20 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD20 as well as any form of CD20 that results from processing in the cell. The term also encompasses naturally occurring variants of CD20, e.g., splice variants or allelic variants. In some aspects, CD20 is human CD20. Human CD20 is described in UniProt (www.uniprot.org) accession no. P11836 (entry version 200), and an amino acid sequence of human CD20 is also shown in SEQ ID NO: 24.

Useful T cell bispecific antibodies for the present invention that bind to CD20 are described e.g. in PCT publication no. WO 2016/020309 (incorporated herein by reference in its entirety).

In some aspects, the T cell bispecific antibody comprises a first antigen binding moiety that binds to CD3, and a second antigen binding moiety that binds to CD20.

In some aspects, the first antigen binding moiety comprises a heavy chain variable region comprising the heavy chain CDR (HCDR) 1 of SEQ ID NO: 4, the HCDR2 of SEQ ID NO: 5, and the HCDR3 of SEQ ID NO: 6; and a light chain variable region comprising the light chain CDR (LCDR) 1 of SEQ ID NO: 7, the LCDR2 of SEQ ID NO: 8 and the LCDR3 of SEQ ID NO: 9.

In some aspects, the second antigen binding moiety comprises a heavy chain variable region comprising the heavy chain CDR (HCDR) 1 of SEQ ID NO: 12, the HCDR2 of SEQ ID NO: 13, and the HCDR3 of SEQ ID NO: 14; and a light chain variable region comprising the light chain CDR (LCDR) 1 of SEQ ID NO: 15, the LCDR2 of SEQ ID NO: 16 and the LCDR3 of SEQ ID NO: 17.

In some aspects, the T cell bispecific antibody comprises
(i) a first antigen binding moiety that binds to CD3 and comprises a heavy chain variable region comprising the heavy chain CDR (HCDR) 1 of SEQ ID NO: 4, the HCDR2 of SEQ ID NO: 5, and the HCDR3 of SEQ ID NO: 6; and a light chain variable region comprising the light chain CDR (LCDR) 1 of SEQ ID NO: 7, the LCDR2 of SEQ ID NO: 8 and the LCDR3 of SEQ ID NO: 9; and
(ii) a second antigen binding moiety that binds to CD20 and comprises a heavy chain variable region comprising the heavy chain CDR (HCDR) 1 of SEQ ID NO: 12, the HCDR2 of SEQ ID NO: 13, and the HCDR3 of SEQ ID NO: 14; and a light chain variable region comprising the light chain CDR (LCDR) 1 of SEQ ID NO: 15, the LCDR2 of SEQ ID NO: 16 and the LCDR3 of SEQ ID NO: 17.

In some aspects, the first antigen binding moiety comprises a heavy chain variable region sequence that is at least about 95%, 96%, 97%, 98%, 99% or 100% identical to the amino acid sequence of SEQ ID NO: 10 and a light chain variable region sequence that is at least about 95%, 96%, 97%, 98%, 99% or 100% identical to the amino acid sequence of SEQ ID NO: 11. In some aspects, the first antigen binding moiety comprises the heavy chain variable region sequence of SEQ ID NO: and the light chain variable region sequence of SEQ ID NO: 11.

In some aspects, the second antigen binding moiety comprises a heavy chain variable region sequence that is at least about 95%, 96%, 97%, 98%, 99% or 100% identical to the amino acid sequence of SEQ ID NO: 18 and a light chain variable region sequence that is at least about 95%, 96%, 97%, 98%, 99% or 100% identical to the amino acid sequence of SEQ ID NO: 19. In some aspects, the second antigen binding moiety comprises the heavy chain variable region sequence of SEQ ID NO: 18 and the light chain variable region sequence of SEQ ID NO: 19.

In some aspects, the T cell bispecific antibody comprises a third antigen binding moiety that binds to CD20 and/or an Fc domain composed of a first and a second subunit, as described herein.

In preferred aspects, the T cell bispecific antibody comprises
(i) a first antigen binding moiety that binds to CD3, comprising a heavy chain variable region comprising the heavy chain CDR (HCDR) 1 of SEQ ID NO: 4, the HCDR2 of SEQ ID NO: 5, and the HCDR3 of SEQ ID NO: 6; and a light chain variable region comprising the light chain CDR (LCDR) 1 of SEQ ID NO: 7, the LCDR2 of SEQ ID NO: 8 and the LCDR3 of SEQ ID NO: 9, wherein the first antigen binding moiety is a crossover Fab molecule wherein either the variable or the constant regions, particularly the variable regions, of the Fab light chain and the Fab heavy chain are exchanged;
(ii) a second and a third antigen binding moiety that bind to CD20, comprising a heavy chain variable region comprising the heavy chain CDR (HCDR) 1 of SEQ ID NO: 12, the HCDR2 of SEQ ID NO: 13, and the HCDR3 of SEQ ID NO: 14; and a light chain variable region comprising the light chain CDR (LCDR) 1 of SEQ ID NO: 15, the LCDR2 of SEQ ID NO: 16 and the LCDR3 of SEQ ID NO: 17, wherein the second and third antigen binding moiety are each a Fab molecule, particularly a conventional Fab molecule;
(iii) an Fc domain composed of a first and a second subunit,
   wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In some aspects, the first antigen binding moiety of the T cell bispecific antibody (that binds to CD20 and CD3) is a crossover Fab molecule wherein the variable regions of the Fab light chain and the Fab heavy chain are exchanged, and wherein the second and (where present) third antigen binding moiety of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

Particularly, in the above aspects, in the constant domain CL of the second and the third Fab molecule under (ii) the amino acid at position 124 may be substituted by lysine (K) (numbering according to Kabat) and the amino acid at position 123 may be substituted by lysine (K) or arginine (R), particularly by arginine (R) (numbering according to Kabat), and in the constant domain CH1 of the second and the third Fab molecule under (ii) the amino acid at position 147 may be substituted by glutamic acid (E) (numbering according to Kabat EU index) and the amino acid at position 213 may be substituted by glutamic acid (E) (numbering according to Kabat EU index).

In some aspects, the first antigen binding moiety of the T cell bispecific antibody (that binds to CD20 and CD3) comprises a heavy chain variable region sequence that is at least about 95%, 96%, 97%, 98%, 99% or 100% identical to the amino acid sequence of SEQ ID NO: 10 and a light chain variable region sequence that is at least about 95%, 96%, 97%, 98%, 99% or 100% identical to the amino acid sequence of SEQ ID NO: 11. In some aspects, the first antigen binding moiety comprises the heavy chain variable region sequence of SEQ ID NO: 10 and the light chain variable region sequence of SEQ ID NO: 11.

In some aspects, the second and (where present) third antigen binding moiety of the T cell bispecific antibody (that binds to CD20 and CD3) comprise a heavy chain variable region sequence that is at least about 95%, 96%, 97%, 98%, 99% or 100% identical to the amino acid sequence of SEQ ID NO: 18 and a light chain variable region sequence that is at least about 95%, 96%, 97%, 98%, 99% or 100% identical to the amino acid sequence of SEQ ID NO: 19. In some aspects, the second and (where present) third antigen binding moiety comprise the heavy chain variable region of SEQ ID NO: 18 and the light chain variable region of SEQ ID NO: 19.

The Fc domain according to the above aspects may incorporate, singly or in combination, all of the features described hereinabove in relation to Fc domains.

In some aspects, the Fc domain of the T cell bispecific antibody (that binds to CD20 and CD3) comprises a modification promoting the association of the first and the second subunit of the Fc domain, and/or the Fc domain comprises one or more amino acid substitution that reduces binding to an Fc receptor and/or effector function.

In some aspects, the antigen binding moieties and the Fc region are fused to each other by peptide linkers, particularly by peptide linkers as in SEQ ID NO: 21 and SEQ ID NO: 23.

In some aspects, the T cell bispecific antibody (that binds to CD20 and CD3) comprises a polypeptide (particularly two polypeptides) comprising a sequence that is at least 80%, 85%, 90%, 95%, 96%, 97%, 98%, or 99% identical to the sequence of SEQ ID NO: 20, a polypeptide comprising a sequence that is at least 80%, 85%, 90%, 95%, 96%, 97%, 98%, or 99% identical to the sequence of SEQ ID NO: 21, a polypeptide comprising a sequence that is at least 80%, 85%, 90%, 95%, 96%, 97%, 98%, or 99% identical to the sequence of SEQ ID NO: 22, and a polypeptide comprising a sequence that is at least 80%, 85%, 90%, 95%, 96%, 97%, 98%, or 99% identical to the sequence of SEQ ID NO: 23. In some aspects, the T cell bispecific antibody (that binds to CD20 and CD3) comprises a polypeptide (particularly two polypeptides) comprising the sequence of SEQ ID NO: 20, a polypeptide comprising the sequence of SEQ ID NO: 21, a polypeptide comprising the sequence of SEQ ID NO: 22, and a polypeptide comprising the sequence of SEQ ID NO: 23.

In preferred aspects, the T cell bispecific antibody is glofitamab (WHO Drug Information (International Nonproprietary Names for Pharmaceutical Substances), Recommended INN: List 83, 2020, vol. 34, no. 1, p. 39).

In some aspects, the disease (to be treated by the T cell engaging agent) is cancer.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “cancer” refers to the physiological condition in mammals that is typically characterized by unregulated cell proliferation. Examples of cancer include but are not limited to, carcinoma, lymphoma, blastoma, sarcoma and leukemia. More non-limiting examples of cancers include haematological cancer such as leukemia, bladder cancer, brain cancer, head and neck cancer, pancreatic cancer, biliary cancer, thyroid cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, sarcoma, bone cancer, and kidney cancer. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases.

In some aspects, the cancer is a cancer expressing the target cell antigen of the T cell engaging agent (e.g. the T cell bispecific antibody).

In some aspects, the cancer is a CD20-expressing cancer (in particular in aspects, wherein the target cell antigen of the T cell engaging agent, e.g. T cell bispecific antibody, is CD20). By “CD20-positive cancer” or “CD20-expressing cancer” is meant a cancer characterized by expression or overexpression of CD20 in cancer cells. The expression of CD20 may be determined for example by quantitative real-time PCR (measuring CD20 mRNA levels), flow cytometry, immunohistochemistry (IHC) or western blot assays. In some aspects, the cancer expresses CD20. In some aspects, the cancer expresses CD20 in at least 20%, preferably at least 50% or at least 80% of tumor cells as determined by immunohistochemistry (IHC) using an antibody specific for CD20.

In some aspects, the cancer is a B-cell cancer, particularly a CD20-positive B-cell cancer (in particular in aspects, wherein the target cell antigen of the T cell engaging agent, e.g. T cell bispecific antibody, is CD20). In some aspects, the cancer is selected from the group consisting of Non-Hodgkin lymphoma (NHL), acute lymphocytic leukemia (ALL), chronic lymphocytic leukemia (CLL), diffuse large B-cell lymphoma (DLBCL), high grade B cell lymphoma (HGBCL), primary mediastinal large B-cell lymphoma (PMBCL), follicular lymphoma (FL), mantle-cell lymphoma (MCL), marginal zone lymphoma (MZL), Multiple myeloma (MM) or Hodgkin lymphoma (HL). In particular aspects, the cancer is selected from the group consisting of Non-Hodgkin lymphoma (NHL), acute lymphocytic leukemia (ALL), chronic lymphocytic leukemia (CLL), diffuse large B-cell lymphoma (DLBCL), high grade B cell lymphoma (HGBCL), primary mediastinal large B-cell lymphoma (PMBCL), follicular lymphoma (FL), mantle-cell lymphoma (MCL) and marginal zone lymphoma (MZL). In some aspects, the cancer is NHL. In some aspects, the NHL is relapsed or refractory (r/r) NHL. In some aspects, the NHL is indolent NHL (iNHL) or aggressive NHL (aNHL). In some aspects, the cancer is DLBCL. In some aspects, the DLBCL is a Richter's transformation. In some aspects, the cancer is high grade B cell lymphoma (HGBCL). In some aspects, the cancer is primary mediastinal large B-cell lymphoma (PMBCL). In some aspects, the cancer is FL. In some aspects, the FL is relapsed or refractory (r/r) FL. In some aspects, the FL is a transformed FL. In some aspects, the cancer is MCL. In some aspects, the MCL is relapsed or refractory (r/r) MCL. In some aspects, the cancer is MZL.

In some aspects, the cancer is treatable by the T cell engaging agent. In some aspects, the T cell engaging agent is indicated for the treatment of the cancer.

In some aspects, the T cell engaging agent (particularly a T cell bispecific antibody that binds to CD20 and CD3, as described hereinabove) is admininstered to the individual in a dosing regimen comprising at least a first dosing cycle and a second dosing cycle, wherein: (a) the first dosing cycle comprises a first dose (C1D1) and a second dose (C1D2) of the T cell engaging agent, wherein the C1D1 is 2.5 mg, and the C1D2 is 10 mg; and (b) the second dosing cycle comprises a single dose (C2D1) of either 16 or 30 mg of the T cell engaging agent. In some aspects, the single dose of the second dosing cycle comprises 30 mg of the T cell engaging agent. In some aspects, the first dose (C1D1) is administered on day 1 of the first dosing cycle and the second dose (C1D2) is administered on day 8 of the first dosing cycle. In some aspects, the single dose of the second dosing cycle (C2D1) is administered on day 1 of the second dosing cycle. In some aspects, the dosing regimen comprises 1 to 10 (C3 to C12) additional dosing cycles. In some such aspects, the 1 to 10 additional dosing cycles (C3 to C12) comprise a single dose (C3D1 to C12D1) of either 16 or 30 mg of the T cell engaging agent. In some aspects, the single dose of the additional dosing cycles (C3D1 to C12D1) comprises 30 mg of the T cell engaging agent. In some aspects, the single dose of the additional dosing cycles (C3D1 to C12D1) is administered on day 1 of the respective additional dosing cycle. In some aspects, the dosing regimen comprises 12 dosing cycles in total. In some aspects, one treatment cycle comprises 14 days or 21 days. In some aspects, one treatment cycle comprises 21 days. In some aspects, treatment with the T cell engaging agent is stopped after 12 dosing cycles.

An “individual” or “subject” herein is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). In certain aspects, the individual or subject is a human. In some aspects, the individual has a disease, particularly a disease treatable or to be treated by the T cell engaging agent. In some aspects, the individual has cancer, particularly a cancer treatable or to be treated by the T cell engaging agent. In particular, an individual herein is any single human subject eligible for treatment who is experiencing or has experienced one or more signs, symptoms, or other indicators of cancer. In some aspects, the individual has cancer or has been diagnosed with cancer, in particular any of the cancers described hereinabove. In some aspects, the individual has locally advanced or metastatic cancer or has been diagnosed with locally advanced or metastatic cancer. The individual may have been previously treated with a T cell engaging agent (e.g. a T cell bispecific antibody) or another drug, or not so treated. In particular aspects, the patient has not been previously treated with a T cell engaging agent (e.g. a T cell bispecific antibody). The patient may have been treated with a therapy comprising one or more drugs other than T cell engaging agent (e.g. other than a T cell bispecific antibody) before the T cell engaging agent therapy is commenced.

In some aspects, the individual has an elevated serum level of one of more cytokine. In some aspects, said elevated serum level is related to the administration of the T cell engaging agent to the individual. Said elevated serum level is in particular as compared to the serum level in a healthy individual, and/or the serum level in an individual (including the same individual) without administration of the T cell engaging agent (i.e. in such case the serum level is elevated as compared to the serum level without administration of the T cell engaging agent). In some aspects, said one or more cytokine is selected from the group consisting of IL-1β, IL-6 and IL-8.

A cytokine according to any of the aspects of the invention may be one or more cytokine selected from the group consisting of interleukin (IL)-1β, IL-6, IL-8, interferon (IFN)-γ, IL-2, tumor necrosis factor (TNF)-α, IP-10 (IFNγ-induced protein 10; also known as C—X—C motif chemokine ligand 10 (CXCL10)), monocyte chemoattractant protein (MCP)-1, interleukin-1 receptor antagonist (IL-1Ra), macrophage-inflammatory protein (MIP)-1α (also known as CCL3) and MIP-(also known as CCL4). In some aspects, the cytokine is one or more cytokine selected from the group consisting of IL-1β, IL-6 and IL-8. In some aspects, the cytokine is IL-1β. In some aspects, the cytokine is IL-6. In some aspects, the cytokine is IL-8. In some aspects, the cytokine is IFN-γ. In some aspects, the cytokine is IL-2. In some aspects, the cytokine is TNF-α. In some aspects, the cytokine is IP-10. In some aspects, the cytokine is MCP-1. In some aspects, the cytokine is IL-1Ra. In some aspects, the cytokine is MIP-1α. In some aspects, the cytokine is MIP-1β.

Preferably, a T cell according to any of the aspects of the invention is a cytotoxic T cell. In some aspects, the T cell is a CD4⁺ or a CD8⁺ T cell. In some aspects, the T cell is a CD8⁺ T cell. In some aspects, the T cell is a CD4⁺ T cell.

In some aspects, the treatment with or administration of the T cell engaging agent may result in a response in the individual. In some aspects, the response may be a complete response. In some aspects, the response may be a sustained response after cessation of the treatment. In some aspects, the response may be a complete response that is sustained after cessation of the treatment. In other aspects, the response may be a partial response. In some aspects, the response may be a partial response that is sustained after cessation of the treatment. In some aspects, the treatment with or administration of the T cell engaging agent and the NLRP3 inhibitor may improve the response as compared to treatment with or administration of the T cell engaging agent alone (i.e. without the NLRP3 inhibitor). In some aspects, the treatment or administration of the T cell engaging agent and the NLRP3 inhibitor may increase response rates in a patient population, as compared to a corresponding patient population treated with the T cell engaging agent alone (i.e. without the NLRP3 inhibitor).

The T cell engaging agent may be used alone or together with other agents in a therapy. For instance, a T cell engaging agent may be co-administered with at least one additional therapeutic agent. In certain aspects, an additional therapeutic agent is an anti-cancer agent, e.g. a chemotherapeutic agent, an inhibitor of tumor cell proliferation, or an activator of tumor cell apoptosis.

The NLRP3 inhibitor may be used alone or together with one or more other agents for the prevention of mitigation of an adverse effect, particularly CRS, related to the administration of the T cell engaging agent. The NLRP3 inhibitor may, for example, be used together with an IL-6R antagonist (e.g. tocilizumab), a steroid (e.g. a corticosteroid such as methylprednisolone, prednisolone and/or dexamethasone) or a TNF-α antagonist (e.g. etanercept).

In some aspects, the NLRP3 inhibitor may be used together with a B-cell depleting agent, such as obinutuzumab (recommended INN, WHO Drug Information, Vol. 26, No. 4, 2012, p. 453). As used herein, obinutuzumab is synonymous for GA101. The tradename is Gazyva® or Gazyvaro®. In some aspects, obinutuzumab is administered as a pre-treatment (“Gazyva® pre-treatment”), before administration of the T cell engaging agent, in particular before the first administration of the T cell engaging agent (e.g. a T cell bispecific antibody that binds to CD20 and CD3, as described hereinabove).

In some aspects, obinutuzumab is administered 1-15 days, particularly 1-10 days, before the (first) administration of the T cell engaging agent. In particular aspects, obinutuzumab is administered 7 days before the (first) administration of the T cell engaging agent.

In some aspects, obinutuzumab is administered at a dose of 1000-2000 mg. In some aspects, obinutuzumab is administered at a single dose of 1000 mg. In some aspects, obinutuzumab is admininstered at a single dose of 2000 mg. In some aspects, obinutuzumab is admininstered at a first and a second dose of 1000 mg each. In some aspects, the first and the second dose of obinutuzumab are administered on the same day, particularly 7 days before the (first) administration of the T cell engaging agent. In particular aspects, 2000 mg obinutuzumab is administered 7 days before the (first) administration of the T cell engaging agent, at a single dose of 2000 mg or a first and a second dose of 1000 mg each. In some aspects, the first and the second dose of obinutuzumab are administered on different days. In particular aspects, the first dose of obinutuzumab is administered 7 days before the (first) administration of the T cell engaging agent, and the second dose of obinutuzumab is administered 1 day before said administration of the T cell engaging agent.

Administration of obinituzumab may be continued during the course of the treatment with the T cell engaging agent (particularly a T cell bispecific antibody that binds to CD20 and CD3, as described hereinabove). In some aspects, obinutuzumab is administered on the first day of the second dosing cycle (C2) and on the first day of any subsequent dosing cycle (e.g. the third (C3) to the twelfth (C12) dosing cycle) with the T cell engaging agent. In some such aspects, obinutuzumab is administered at a dose of 1000 mg. In some aspects, treatment is stopped after a total of 12 dosing cycles with the T cell engaging agent.

Amino Acid Sequences

| | | SEQ |
|---|---|---|
| | Sequence | ID NO |
| Human CD3 | MQSGTHWRVLGLCLLSVGVWGQDGNEEMGGITQTPYKVSISGTTVILTCPQYP | 1 |
| | GSEILWQHNDKNIGGDEDDKNIGSDEDHLSLKEFSELEQSGYYVCYPRGSKPE | |
| | DANFYLYLRARVCENCMEMDVMSVATIVIVDICITGGLLLLVYYWSKNRKAKA | |
| | KPVTRGAGAGGRQRGQNKERPPPVPNPDYEPIRKGQRDLYSGLNQRRI | |
| Cynomolgus | MQSGTRWRVLGLCLLSIGVWGQDGNEEMGSITQTPYQVSISGTTVILTCSQHL | 2 |
| CD3 | GSEAQWQHNGKNKEDSGDRLFLPEFSEMEQSGYYVCYPRGSNPEDASHHLYLK | |
| | ARVCENCMEMDVMAVATIVIVDICITLGLLLLVYYWSKNRKAKAKPVTRGAGA | |
| | GGRQRGQNKERPPPVPNPDYEPIRKGQQDLYSGLNQRRI | |
| hIgG1 Fc | DKTHTCPPCPAPELLGGPSVFLFPPKPKDTLMISRTPEVTCVVVDVSHEDPEV | 3 |
| region | KFNWYVDGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYPSDIAV | |
| | EWESNGQPENNYKTTPPVLDSDGSFFLYSKLTVDKSRWQQGNVFSCSVMHEAL | |
| | HNHYTQKSLSLSP | |
| CD3 HCDR1 | TYAMN | 4 |
| CD3 HCDR2 | RIRSKYNNYATYYADSVKG | 5 |
| CD3 HCDR3 | HGNFGNSYVSWFAY | 6 |
| CD3 LCDR1 | GSSTGAVTTSNYAN | 7 |
| CD3 LCDR2 | GTNKRAP | 8 |
| CD3 LCDR3 | ALWYSNLWV | 9 |
| CD3 VH | EVOLLESGGGLVQPGGSLRLSCAASGFTESTYAMNWVRQAPGKGLEWVSRIRS | 10 |
| | KYNNYATYYADSVKGRFTISRDDSKNTLYLQMNSLRAEDTAVYYCVRHGNFGN | |
| | SYVSWFAYWGQGTLVTVSS | |
| CD3 VL | QAVVTQEPSLTVSPGGTVTLTCGSSTGAVTTSNYANWVQEKPGQAFRGLIGGT | 11 |
| | NKRAPGTPARFSGSLLGGKAALTLSGAQPEDEAEYYCALWYSNLWVFGGGTKL | |
| | TVL | |
| CD20 HCDR1 | YSWIN | 12 |
| CD20 HCDR2 | RIFPGDGDTDYNGKFKG | 13 |
| CD20 HCDR3 | NVFDGYWLVY | 14 |
| CD20 LCDR1 | RSSKSLLHSNGITYLY | 15 |
| CD20 LCDR2 | QMSNLVS | 16 |
| CD20 LCDR3 | AQNLELPYT | 17 |
| CD20 VH | QVQLVQSGAEVKKPGSSVKVSCKASGYAFSYSWINWVRQAPGQGLEWMGRIFP | 18 |
| | GDGDTDYNGKFKGRVTITADKSTSTAYMELSSLRSEDTAVYYCARNVEDGYWL | |
| | VYWGQGTLVTVSS | |
| CD20 VL | DIVMTQTPLSLPVTPGEPASISCRSSKSLLHSNGITYLYWYLQKPGQSPQLLI | 19 |
| | YQMSNLVSGVPDRESGSGSGTDFTLKISRVEAEDVGVYYCAQNLELPYTFGGG | |
| | TKVEIK | |
| CD20 VL- | DIVMTQTPLSLPVTPGEPASISCRSSKSLLHSNGITYLYWYLQKPGQSPQLLI | 20 |
| CL(RK) | YQMSNLVSGVPDRFSGSGSGTDFTLKISRVEAEDVGVYYCAQNLELPYTFGGG | |
| | TKVEIKRTVAAPSVFIFPPSDRKLKSGTASVVCLLNNFYPREAKVQWKVDNAL | |
| | QSGNSQESVTEQDSKDSTYSLSSTLTLSKADYEKHKVYACEVTHQGLSSPVTK | |
| | SENRGEC | |
| CD20 VH- | QVQLVQSGAEVKKPGSSVKVSCKASGYAFSYSWINWVRQAPGQGLEWMGRIFP | 21 |
| CH1(EE)- | GDGDTDYNGKFKGRVTITADKSTSTAYMELSSLRSEDTAVYYCARNVEDGYWL | |
| Fc(hole, | VYWGQGTLVTVSSASTKGPSVFPLAPSSKSTSGGTAALGCLVEDYFPEPVTVS | |
| PGLALA) | WNSGALTSGVHTFPAVLQSSGLYSLSSVVTVPSSSLGTQTYICNVNHKPSNTK | |
| | VDEKVEPKSCDKTHTCPPCPAPEAAGGPSVFLFPPKPKDTLMISRTPEVTCVV | |
| | VDVSHEDPEVKENWYVDGVEVHNAKTKPREEQYNSTYRVVSVLTVLHQDWLNG | |
| | KEYKCKVSNKALGAPIEKTISKAKGQPREPQVCTLPPSRDELTKNQVSLSCAV | |
| | KGFYPSDIAVEWESNGQPENNYKTTPPVLDSDGSFFLVSKLTVDKSRWQQGNV | |
| | FSCSVMHEALHNHYTQKSLSLSP | |
| CD3 VH-CL | EVQLLESGGGLVQPGGSLRLSCAASGFTESTYAMNWVRQAPGKGLEWVSRIRS | 22 |
| | KYNNYATYYADSVKGRFTISRDDSKNTLYLQMNSLRAEDTAVYYCVRHGNFGN | |
| | SYVSWFAYWGQGTLVTVSSASVAAPSVFIFPPSDEQLKSGTASVVCLLNNFYP | |
| | REAKVQWKVDNALQSGNSQESVTEQDSKDSTYSLSSTLTLSKADYEKHKVYAC | |
| | EVTHQGLSSPVTKSENRGEC | |
| CD20 VH- | QVQLVQSGAEVKKPGSSVKVSCKASGYAFSYSWINWVRQAPGQGLEWMGRIFP | 23 |
| CH1(EE)-CD3 | GDGDTDYNGKFKGRVTITADKSTSTAYMELSSLRSEDTAVYYCARNVEDGYWL | |
| VL-CH1- | VYWGQGTLVTVSSASTKGPSVFPLAPSSKSTSGGTAALGCLVEDYFPEPVTVS | |
| Fc(knob, | WNSGALTSGVHTFPAVLQSSGLYSLSSVVTVPSSSLGTQTYICNVNHKPSNTK | |
| PGLALA) | VDEKVEPKSCDGGGGSGGGGSQAVVTQEPSLTVSPGGTVTLTCGSSTGAVTTS | |
| | NYANWVQEKPGQAFRGLIGGTNKRAPGTPARFSGSLLGGKAALTLSGAQPEDE | |
| | AEYYCALWYSNLWVFGGGTKLTVLSSASTKGPSVFPLAPSSKSTSGGTAALGC | |
| | LVKDYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPKPKDT | |
| | LMISRTPEVTCVVVDVSHEDPEVKENWYVDGVEVHNAKTKPREEQYNSTYRVV | |
| | SVLTVLHQDWLNGKEYKCKVSNKALGAPIEKTISKAKGQPREPQVYTLPPCRD | |
| | ELTKNQVSLWCLVKGFYPSDIAVEWESNGQPENNYKTTPPVLDSDGSFFLYSK | |
| | LTVDKSRWQQGNVFSCSVMHEALHNHYTQKSLSLSP | |
| Human CD20 | MTTPRNSVNGTFPAEPMKGPIAMQSGPKPLERRMSSLVGPTQSFFMRESKTLG | 24 |
| | AVQIMNGLFHIALGGLLMIPAGIYAPICVTVWYPLWGGIMYIISGSLLAATEK | |
| | NSRKCLVKGKMIMNSLSLFAAISGMILSIMDILNIKISHFLKMESLNFIRAHT | |
| | PYINIYNCEPANPSEKNSPSTQYCYSIQSLFLGILSVMLIFAFFQELVIAGIV | |
| | ENEWKRTCSRPKSNIVLLSAEEKKEQTIEIKEEVVGLTETSSQPKNEEDIEII | |
| | PIQEEEEEETETNFPEPPQDQESSPIENDSSP | |

### BRIEF DESCRIPTION OF THE DRAWINGS

FIGS. 1A-1N. Effect of the NLRP3 inhibitor on CD20-TCB-mediated T cell activation, B cell depletion and cytokine release. Fresh whole blood from a healthy donor (donor 1) was incubated with escalating doses of CD20-TCB and 10 μM NLRP3 inhibitor. At 24 hrs, blood from technical replicates was pooled and lysed. FIG. 1A. The killing of CD19+ B cells was measured by flow cytometry. FIG. 1B and FIG. 1C. The expression of CD25 on CD4+ (1B) and CD8+ (1C) T cells was measured by flow cytometry. At 24 hrs, serum from technical replicates were collected and pooled. FIG. 1D-FIG. 1N. The levels of IFN-γ (1D), IL-2 (1E), IL-1β (1F), IL-1Ra (1G), TNF-α (1H), IP-10 (1I), IL-6 (1J), IL-8 (1K), MCP-1 (1L), MIP-1α (1M) and MIP-1β (1N) were measured by Luminex.

FIGS. 2A-2N. Effect of the NLRP3 inhibitor on CD20-TCB-mediated T cell activation, B cell depletion and cytokine release. Fresh whole blood from a healthy donor (donor 2) was incubated with escalating doses of CD20-TCB and 10 μM NLRP3 inhibitor. At 24 hrs, blood from technical replicates was pooled and lysed. FIG. 2A. The killing of CD19+ B cells was measured by flow cytometry. FIG. 2B and FIG. 2C. The expression of CD25 on CD4+ (FIG. 2B) and CD8+ (FIG. 2C) T cells was measured by flow cytometry. At 24 hrs, serum from technical replicates were collected and pooled. FIG. 2D-FIG. 2N. The levels of IFN-γ (2D), IL-2 (2E), IL-1β (2F), IL-1Ra (2G), TNF-α (2H), IP-10 (2I), IL-6 (2J), IL-8 (2K), MCP-1 (2L), MIP-1α (2M) and MIP-1β (2N) were measured by Luminex.

FIGS. 3A-3N. Effect of the NLRP3 inhibitor on CD20-TCB-mediated T cell activation, B cell depletion and cytokine release. Fresh whole blood from a healthy donor (donor 3) was incubated with escalating doses of CD20-TCB and 10 NM NLRP3 inhibitor. At 24 hrs, blood from technical replicates was pooled and lysed. FIG. 3A. The killing of CD19+ B cells was measured by flow cytometry. FIG. 3B and FIG. 3C. The expression of CD25 on CD4+ (3B) and CD8+ (3C) T cells was measured by flow cytometry. At 24 hrs, serum from technical replicates were collected and pooled. FIG. 3D-FIG. 3N. The levels of IFN-γ (3D), IL-2 (3E), IL-1β (3F), IL-1Ra (3G), TNF-α (3H), IP-10 (3I), IL-6 (3J), IL-8 (3K), MCP-1 (3L), MIP-1α (3M) and MIP-1β (3N) were measured by Luminex.

FIGS. 4A-4B. Effect of the NLRP3 inhibitor on CD20-TCB-mediated anti-tumor efficacy. OCI-Ly18-bearing humanized NSG mice were pre-treated with 30 mg/kg obinutuzumab (Gazyva® pre-treatment, Gpt, dotted black arrow) and then treated with escalating doses of CD20-TCB (black arrows, 0.5 mg/kg on the 1^{st} treatment, 1 mg/kg on the 2^{nd} treatment and 2 mg/kg on the 3^{rd} treatment) by intravenous (i.v.) injections. 20 mg/kg NLRP3 inhibitor (grey arrows) was given by intraperitoneal injections 1 hr before, 4 hrs and 24 hrs after the 1^{st} CD20-TCB therapy as well as 1 hr before and 4 hrs after the 2nd CD20-TCB therapy. FIG. 4A. Tumor growth inhibition curves, means−/+SD. Tumor volumes were calculated from caliper measurements conducted 2 or 3 times per week. FIG. 4B. Intra-tumoral CD8+ T cell counts 24 hrs after the 3^{rd} CD20-TCB therapy, box plot representing minimum to maximum values for n=5 mice. Tumors were digested and the counts of CD8+ T cells were measured by flow cytometry.

FIGS. 5A-5E. Effect of the NLRP3 inhibitor on IL-2, IFN-γ, TNF-α, IL-1β, and IL-6 induced by CD20-TCB. Treatment was performed as described in FIG. 4. Blood was collected by tail-vein bleedings 3 hrs after the 1^{st} CD20-TCB therapy. The levels of FIG. 5A. IL-2, FIG. 5B. IFN-γ, FIG. 5C. TNF-α, FIG. 5D. IL-1β, and FIG. 5E. IL-6 were measured in the serum by Luminex. Means of n=5 mice+/−SEM.

FIG. 6. Heatmap representing the effects of the NLRP3 inhibitor on CD20-TCB mediated cytokine release. Treatment was performed as described in FIG. 4. Blood was collected by tail-vein bleeding 3 hrs after the 1^{st} CD20-TCB therapy. The levels of IFN-γ, IL-2, TNF-α, IP-10, IL-1β, IL-1Ra, IL-6, IL-8, MCP-1, MIP-1α and MIP-1β were measured in serum by Luminex for n=5 mice per group.

FIG. 7. Effect of the NLRP3 inhibitor on body weight loss induced by the first CD20-TCB therapy. Treatment was performed as described in FIG. 4. The body weight of each animal was measured before and after the first therapy with CD20-TCB to calculate body weight change [%]. Means+/−SEM.

### EXAMPLES

The following are examples of methods and compositions of the invention. It is understood that various other aspects may be practiced, given the general description provided above.

### Example 1. The NLRP3 Inhibitor Prevents TCB-Mediated Cytokine Release with Minimal Impact on T Cell Activation and T Cell Cytotoxicity

To assess the effects of the NLRP3 inhibitor on TCB-mediated target cell killing and T cell activation, we conducted whole blood assays using fresh whole blood from 3 healthy donors incubated with escalating concentrations of CD20-TCB (SEQ ID NOs 4-23) in combination with 10 μM of the NLRP3 inhibitor (MCC950, Sigma-Aldrich). At 24 hrs, the killing of CD19+ B cells together with the expression of CD25 on CD4+ and CD8+ T cells was measured in whole blood by flow cytometry.

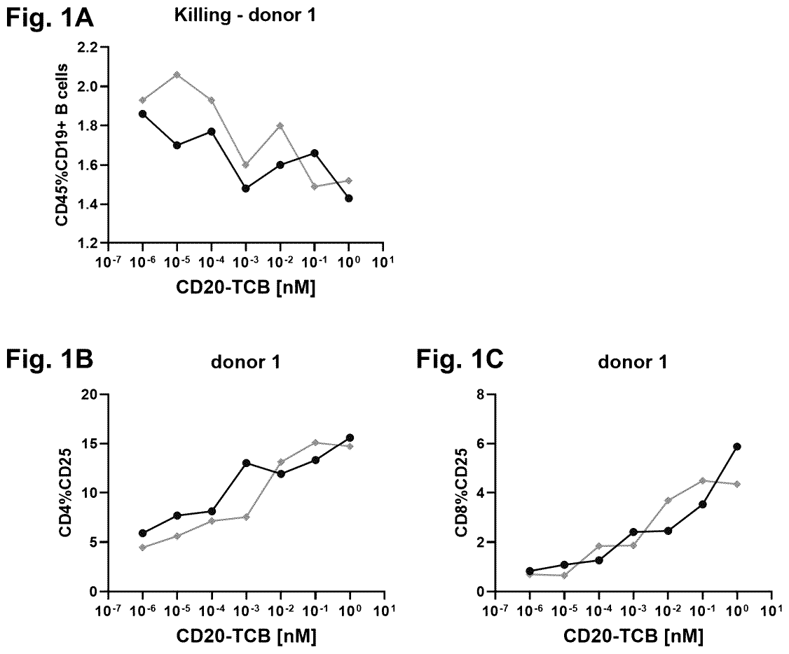

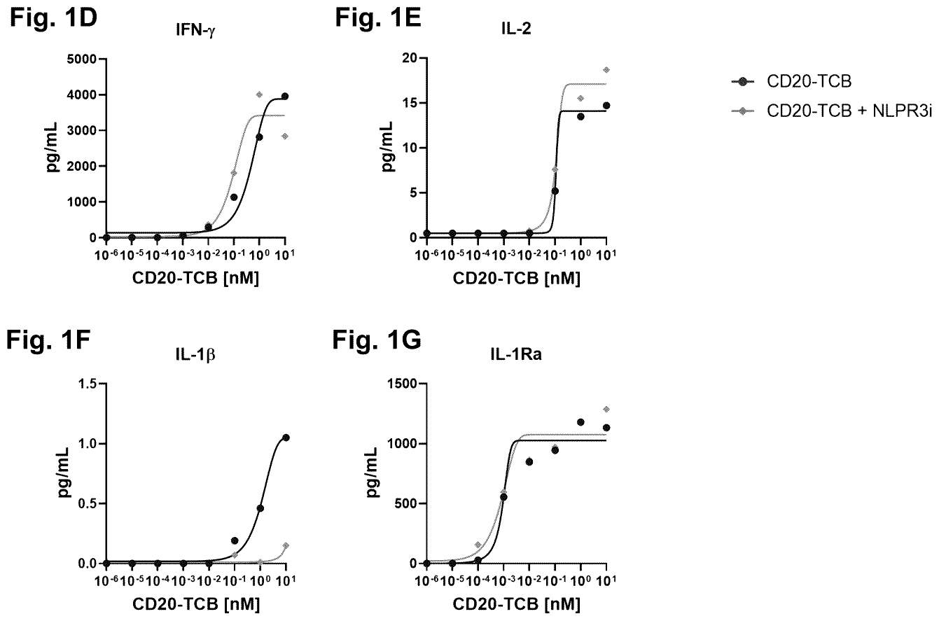

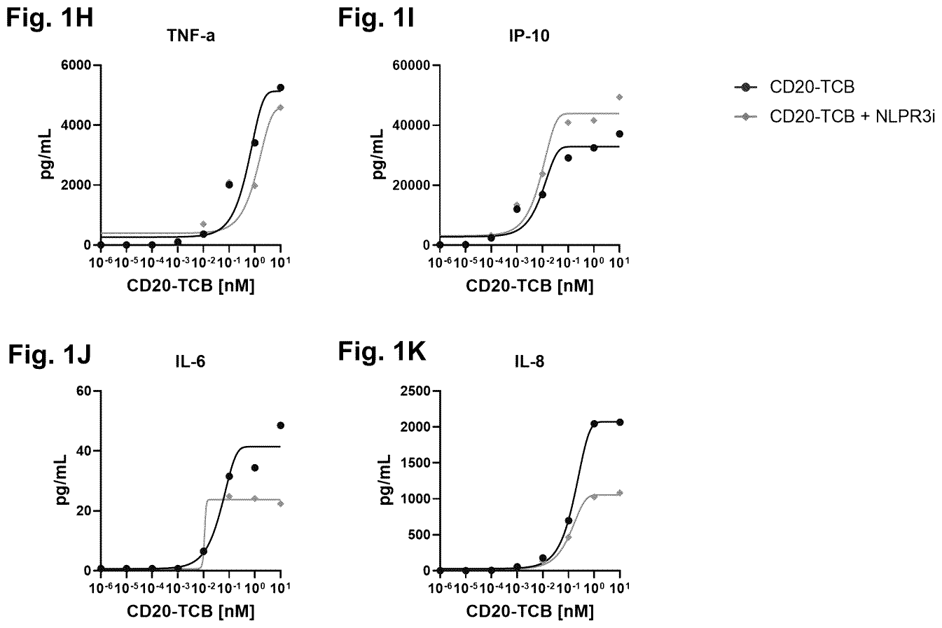

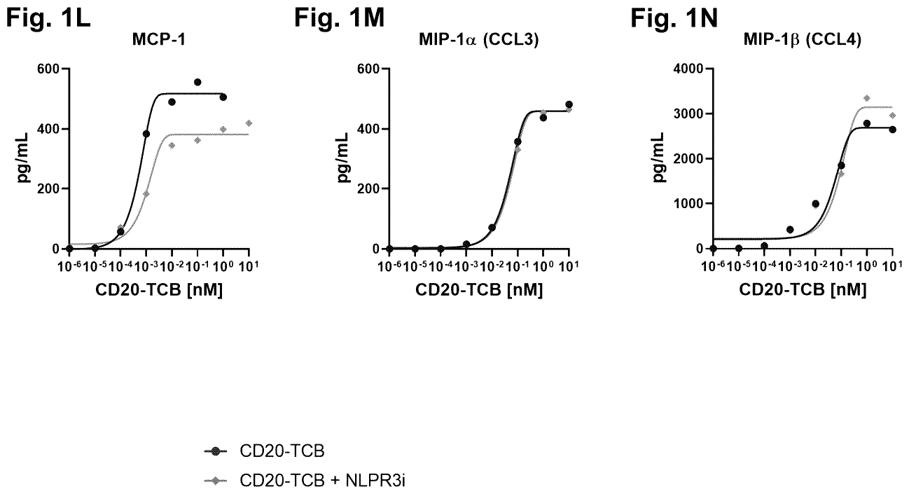

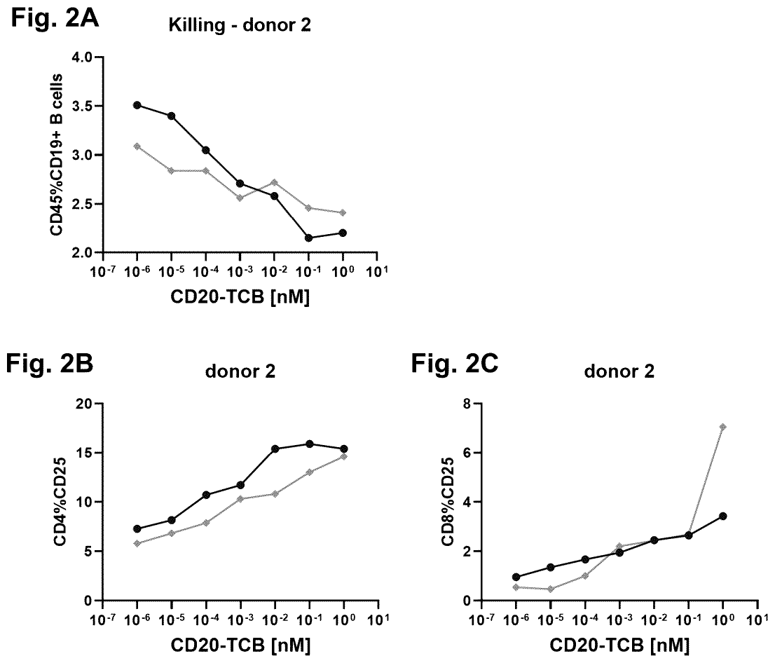

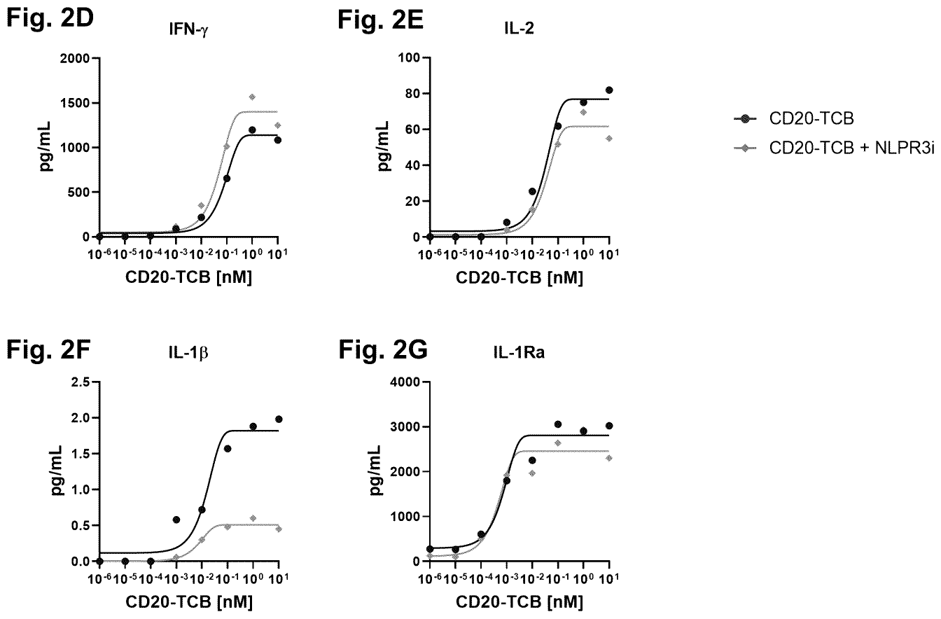

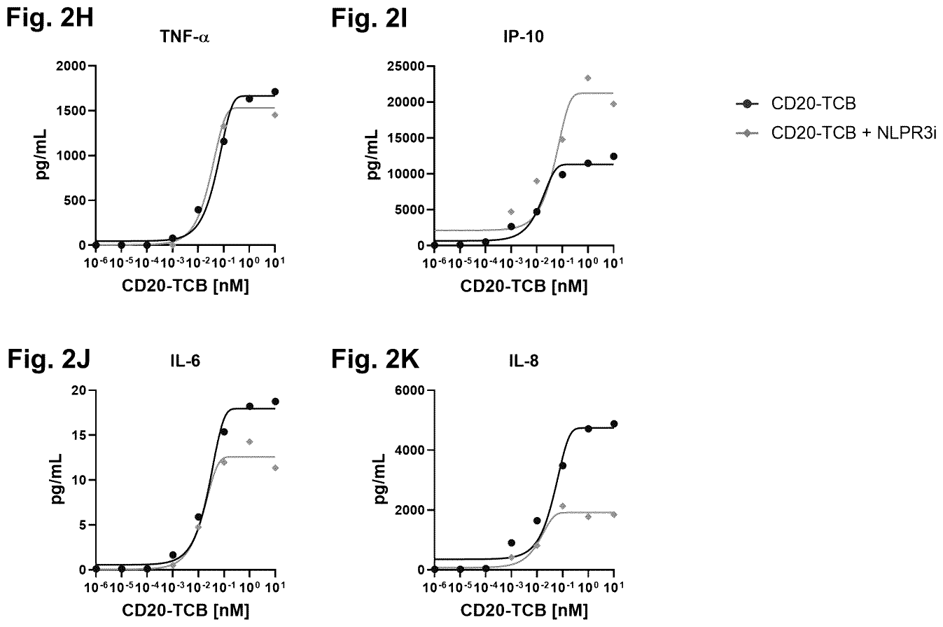

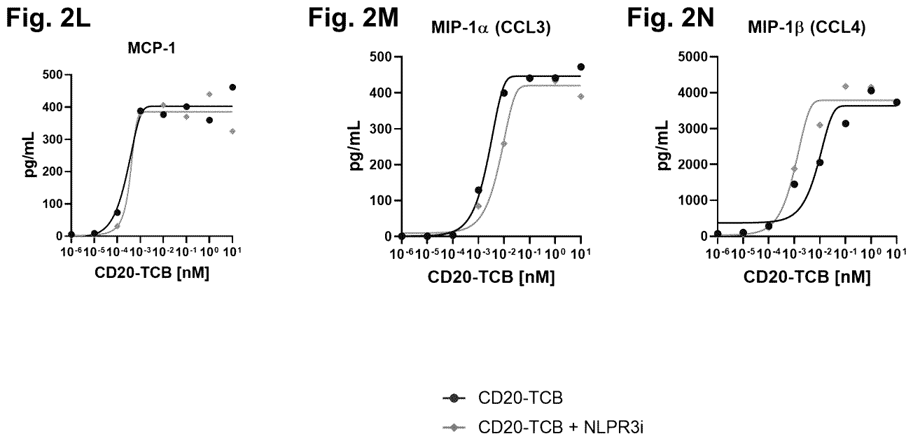

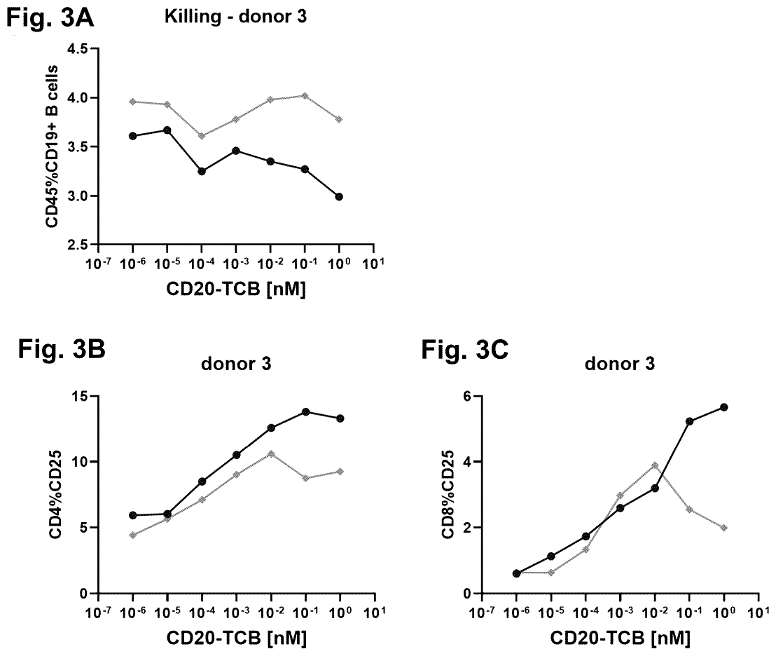

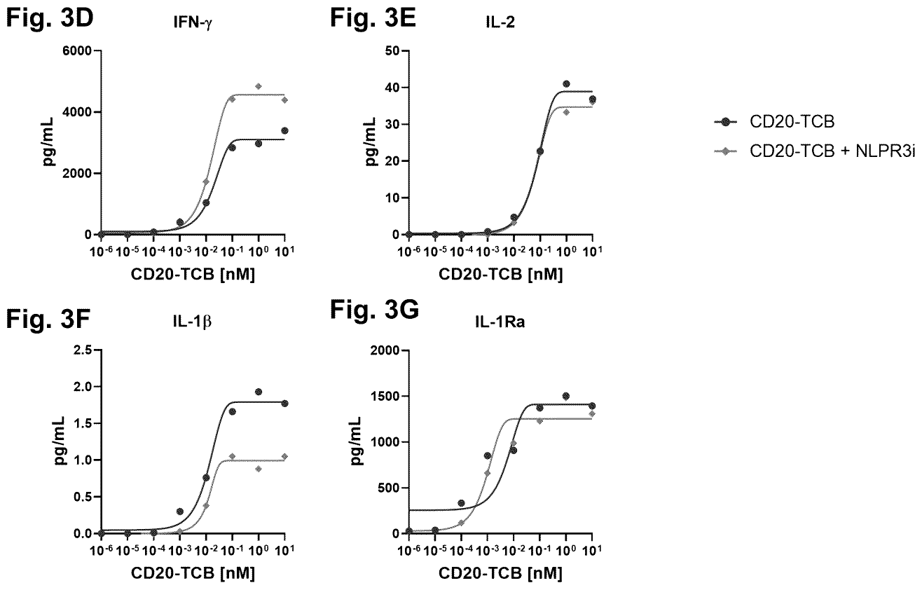

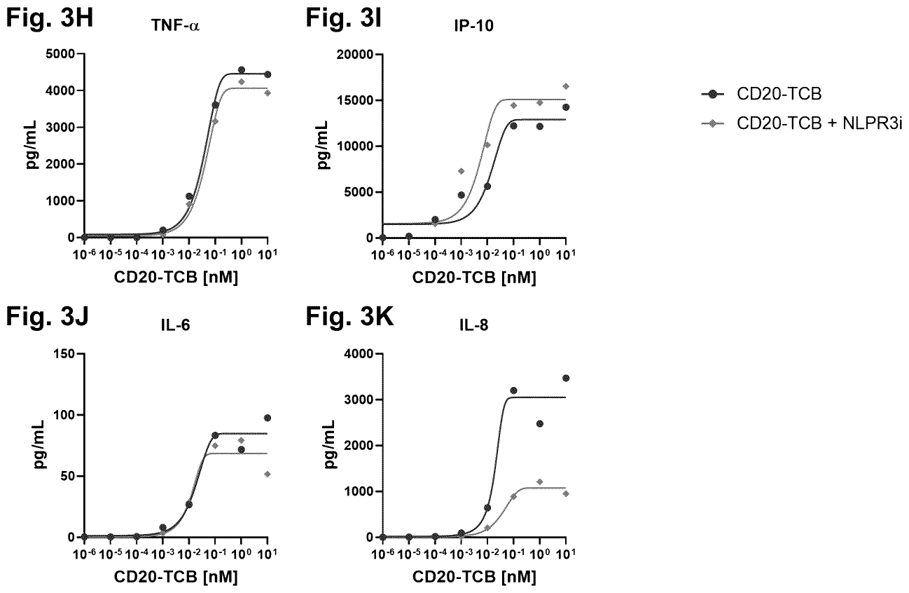

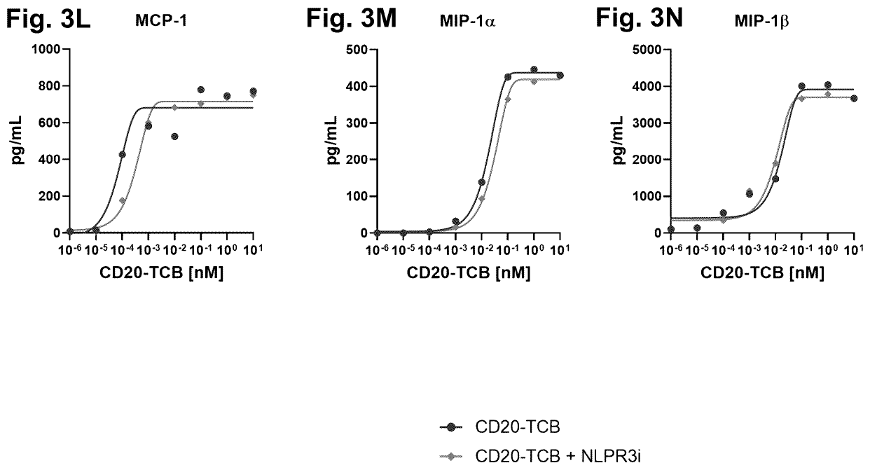

The NLRP3 inhibitor minimally interfered with CD20-TCB-induced B cell depletion, as indicated by the percentage of CD19+ B cells among CD45+ cells (FIG. 1A, FIG. 2A and FIG. 3A). In line with this, the NLRP3 inhibitor did not prevent the expression of the activation marker CD25 on CD4+ and CD8+ T cells, indicating that it did not interfere with T cell activation induced by CD20-TCB. (FIGS. 1 B and C, FIGS. 2 B and C, and FIGS. 3 B and C).

The levels of cytokines were measured by Luminex in the serum of whole blood treated with CD20-TCB in the presence and in the absence of the NLRP3 inhibitor to determine the effects of the NLRP3 inhibitor on cytokine release. As a result, the NLRP3 inhibitor reduced the levels of IL-1β, and IL-8 in all 3 donors, and of IL-6 in 2 out of 3 donors. However, it did not influence the levels of IFN-γ, IL-2, TNF-α, IP-10, IL-1Ra, MCP-1, MIP-1α and MIP-10 (FIGS. 1 D-N, FIGS. 2 D-N, and FIGS. 3 D-N).

In summary, the NLRP3 inhibitor retained T cell cytotoxicity and T cell activation following stimulation with CD20-TCB while reducing the release of IL-6, IL-1β and IL-8. Given the contribution of these cytokines in the pathophysiology of CRS, this data suggests that an NLRP3 inhibitor may be an attractive compound for the mitigation of CRS in the context of T cell engaging therapies.

### Example 2. The NLRP3 Inhibitor Retains In Vivo Efficacy while Preventing TCB-Mediated Cytokine Release and Body Weight Loss

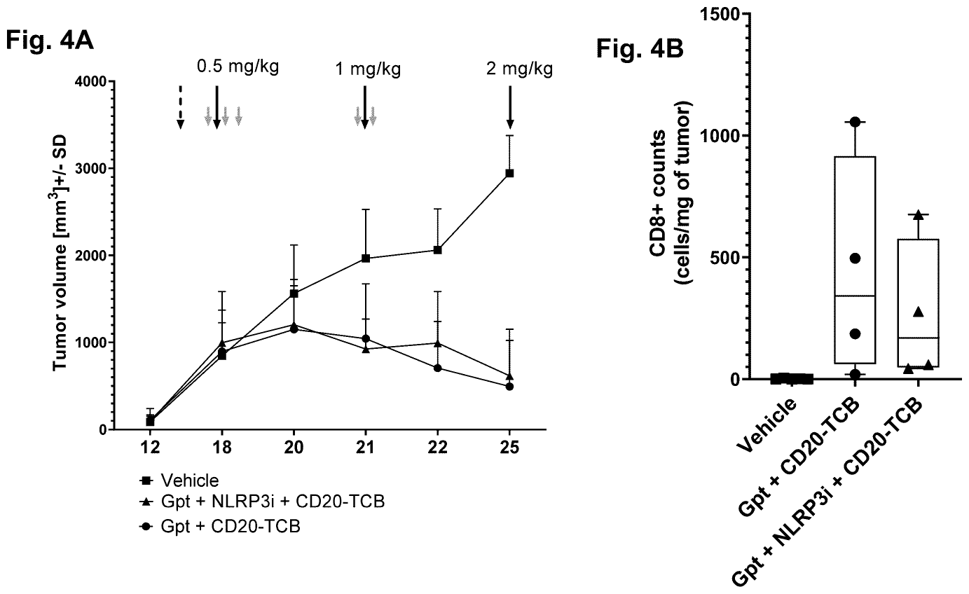

The effects of the NLRP3 inhibitor on anti-tumor efficacy and cytokine release mediated by CD20-TCB in vivo were evaluated using a diffused large B cell lymphoma (DLBCL) tumor model in humanized NSG mice. Briefly, humanized NSG mice were engrafted with OCI-LY18 tumor cells. When the tumor volume reached 200 mm³, mice were randomized in groups of 14 based on their tumor size. They were then pre-treated with 30 mg/kg obinutuzumab (Gazyva® pre-treatment, GpT) 7 days before being treated with CD20-TCB to deplete peripheral B cells. Mice received weekly intravenous injections with 0.5 mg/kg, 1 mg/kg and 2 mg/kg CD20-TCB as shown in FIG. 4A. The NLRP3 inhibitor was given 1 hr before and 4 and 24 hrs after the first CD20-TCB treatment as well as 1 hr before and 4 hrs after the second CD20-TCB treatment.

To assess the effects of the NLRP3 inhibitor on tumor growth, tumor volumes were calculated from caliper measurements twice or three times per week. Furthermore, the counts of CD8+ T cells were measured by flow cytometry in tumors collected 24 hrs after the third treatment with CD20-TCB. As a result, the NLRP3 inhibitor did not interfere with anti-tumor efficacy mediated by CD20-TCB (FIG. 4A) nor with CD8+ T cell infiltration in tumors (FIG. 4B).

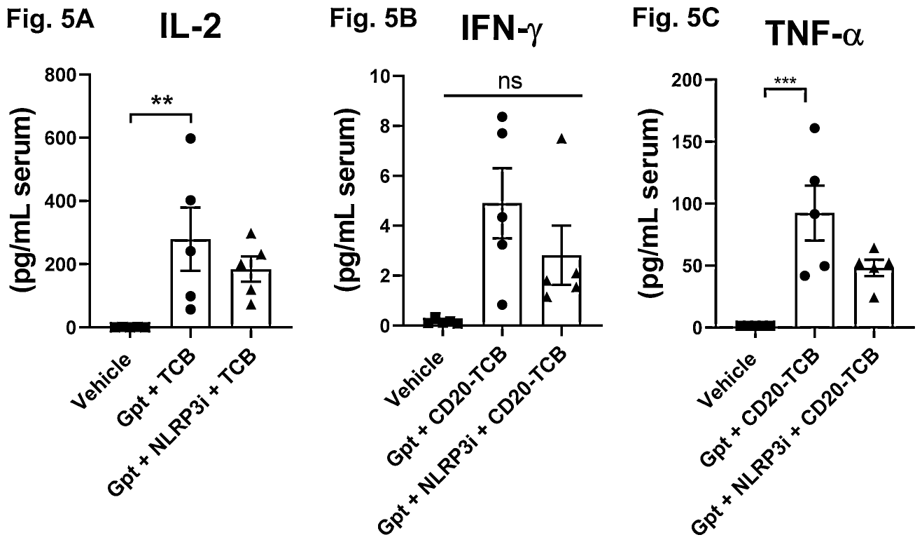

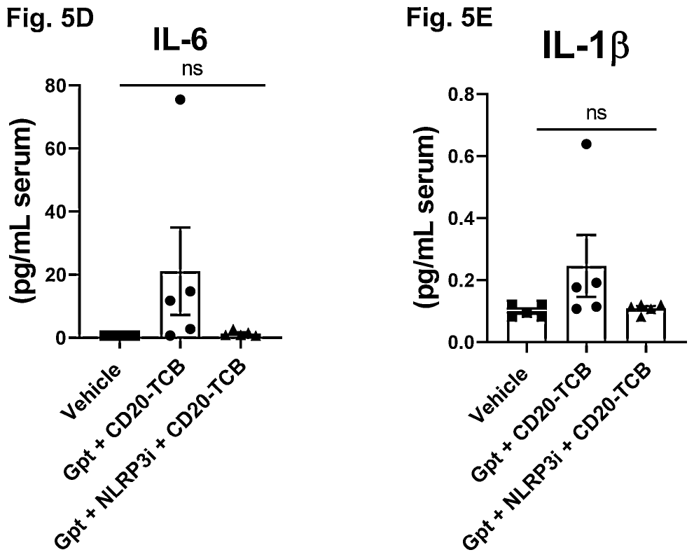

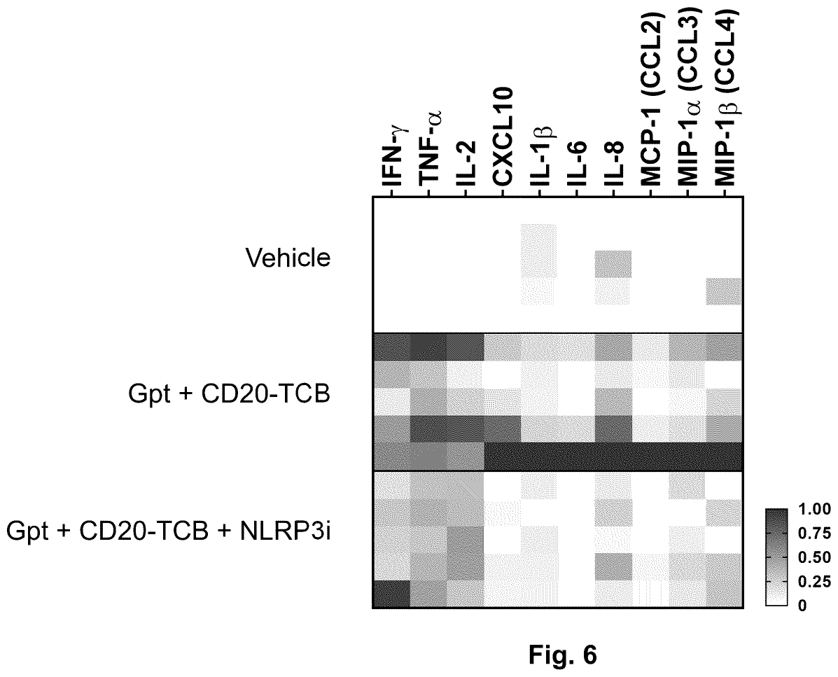

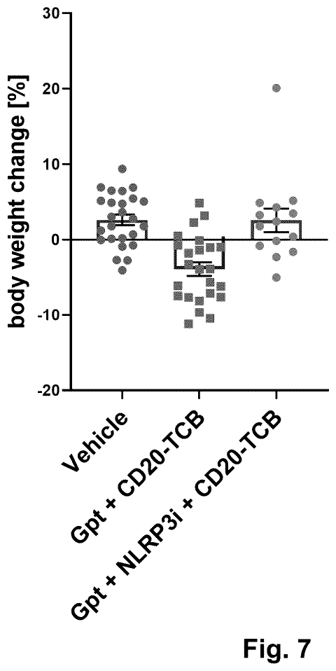

The levels of cytokines were measured in serum collected by tail-vein bleedings 3 hrs after the first treatment with CD20-TCB to determine the effects of the NLRP3 inhibitor on CD20-TCB-mediated cytokine release in vivo. In line with previous in vitro findings, the NLRP3 inhibitor retained IL-2, IFN-γ, TNF-α while reducing IL-6, IL-8 and IL-1β levels induced by CD20-TCB (FIGS. 5A-E, FIG. 6). In addition, the NLRP3 inhibitor modified the cytokine profile induced by CD20-TCB treatment, as indicated by the heat map showing the levels of IFN-γ, IL-2, TNF-α, IP-IL-10, IL-1Ra, IL-6, IL-8, MCP-1, MIP-1α and MIP-10 which were measured 3 hrs after the first treatment with CD20-TCB (FIG. 6). Last, we measured the body weight of each animal before and after the first treatment with CD20-TCB to calculate the body weight change. We found that the NLRP3 inhibitor prevented body weight loss induced by CD20-TCB, suggesting that it may prevent CRS clinical signs. (FIG. 7).

In summary, the NLRP3 inhibitor retained CD20-TCB anti-tumor activity while reducing the levels of IL-1β and IL-6. This resulted in preventing body weight loss induced by the first treatment with CD20-TCB. Altogether, this data support the use of an NLRP3 inhibitor for the mitigation of CRS symptoms that may be induced by treatment with TCBs.

### ***

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1.-2. (canceled)

3. A method for treatment of a disease in an individual, wherein said method comprises
(a) the administration of a T cell engaging agent to the individual, and
(b) the administration of an NLRP3 inhibitor to the individual.

4. The method of claim 3, wherein the administration of the NLRP3 inhibitor prevents or mitigates an adverse effect related to the administration of the T cell engaging agent.

5.-6. (canceled)

7. A method for preventing or mitigating an adverse effect related to the administration of a T cell engaging agent to an individual, comprising the administration of an NLRP3 inhibitor to the individual.

8. The method of claim 3 or 7, wherein the NLRP3 inhibitor is MCC950, or a derivative thereof.

9. The method of claim 3 or 7, wherein the NLRP3 inhibitor is selected from the group consisting of MCC950, selnoflast, emlenoflast, CY-09, IZD334, IZD174, DFV890, IFM-632, IFM-514, JT194, JT349, NT-0167, NT-0796, NT-0249, VENT-01, VTX3232, VTX2735, BT032, BT132, OLT1177, ADS-032, ZYIL1, HY209, AC-201, AC-203, and tranilast.

10. The method of claim 3 or 7, wherein the administration of the NLRP3 inhibitor inhibits an adverse effect related to the administration of the T cell engaging agent.

11. The method of claim 3 or 7, wherein the administration of the NLRP3 inhibitor does not inhibit a desired effect related to the administration of the T cell engaging agent.

12. The method of claim 10, wherein the inhibition is a complete inhibition, or a clinically meaningful and/or statistically significant inhibition.

13. The method of claim 3 or 7, wherein the adverse effect is one or more adverse effect selected from the group consisting of:
(i) cytokine release syndrome (CRS);
(ii) fever, hypotension and/or hypoxia; and
(iii) an elevated serum level of one of more cytokine.

14. The method of claim 3 or 7, wherein the administration of the NLRP3 inhibitor is upon manifestation of the adverse effect in the individual.

15. The method of claim 3 or 7, wherein the administration of the NLRP3 inhibitor is
(i) before, concurrent to, and/or after the administration of the T cell engaging agent;
(ii) intermittent or continuous; and/or
(iii) oral or parenteral.

16. The method of claim 3 or 7, wherein the administration of the NLRP3 inhibitor is associated with the first administration of the T cell engaging agent, wherein the administration of the NLRP3 inhibitor is prior to, concurrent with, and/or subsequent to the first administration of the T cell engaging agent.

17. The method of claim 3 or 7, wherein the administration of the T cell engaging agent is
(i) at an effective dose;
(ii) parenteral; and/or
(iii) the first administration of the T cell engaging agent to the individual.

18. The method of claim 3 or 7, wherein the T cell engaging agent is a T cell bispecific antibody or a CAR-T cell.

19. The method of claim 18, wherein the T cell bispecific antibody binds to CD3 and a target cell antigen.

20. The method of claim 19, wherein the T cell bispecific antibody comprises an antigen binding moiety that binds to CD3 and an antigen binding moiety that binds to a target cell antigen.

21. The method of claim 20, wherein the target cell antigen is CD20.

22. The method of claim 21, wherein the T cell bispecific antibody comprises
(i) a first antigen binding moiety that binds to CD3 and comprises a heavy chain variable region comprising the heavy chain CDR (HCDR) 1 of SEQ ID NO: 4, the HCDR2 of SEQ ID NO: 5, and the HCDR3 of SEQ ID NO: 6; and a light chain variable region comprising the light chain CDR (LCDR) 1 of SEQ ID NO: 7, the LCDR2 of SEQ ID NO: 8 and the LCDR3 of SEQ ID NO: 9; and
(ii) a second antigen binding moiety that binds to CD20 and comprises a heavy chain variable region comprising the heavy chain CDR (HCDR) 1 of SEQ ID NO: 12, the HCDR2 of SEQ ID NO: 13, and the HCDR3 of SEQ ID NO: 14; and a light chain variable region comprising the light chain CDR (LCDR) 1 of SEQ ID NO: 15, the LCDR2 of SEQ ID NO: 16 and the LCDR3 of SEQ ID NO: 17.

23. The method of claim 21, wherein the T cell bispecific antibody comprises a third antigen binding moiety that binds to CD20 and/or an Fc domain composed of a first and a second subunit.

24. The method of claim 23, wherein the T cell bispecific antibody comprises
(i) a first antigen binding moiety that binds to CD3, comprising a heavy chain variable region comprising the heavy chain CDR (HCDR) 1 of SEQ ID NO: 4, the HCDR2 of SEQ ID NO: 5, and the HCDR3 of SEQ ID NO: 6; and a light chain variable region comprising the light chain CDR (LCDR) 1 of SEQ ID NO: 7, the LCDR2 of SEQ ID NO: 8 and the LCDR3 of SEQ ID NO: 9,
wherein the first antigen binding moiety is a crossover Fab molecule wherein either the variable or the constant regions of the Fab light chain and the Fab heavy chain are exchanged;
(ii) a second and a third antigen binding moiety that bind to CD20, comprising a heavy chain variable region comprising the heavy chain CDR (HCDR) 1 of SEQ ID NO: 12, the HCDR2 of SEQ ID NO: 13, and the HCDR3 of SEQ ID NO: 14; and a light chain variable region comprising the light chain CDR (LCDR) 1 of SEQ ID NO: 15, the LCDR2 of SEQ ID NO: 16 and the LCDR3 of SEQ ID NO: 17,
wherein the second and third antigen binding moiety are each a Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

25. The method of claim 24, wherein the first antigen binding moiety of the T cell bispecific antibody comprises a heavy chain variable region sequence that is at least about 95%, 96%, 97%, 98%, 99% or 100% identical to the amino acid sequence of SEQ ID NO: 10 and a light chain variable region sequence that is at least about 95%, 96%, 97%, 98%, 99% or 100% identical to the amino acid sequence of SEQ ID NO: 11,
and/or the second and third antigen binding moiety of the T cell bispecific antibody comprise a heavy chain variable region sequence that is at least about 95%, 96%, 97%, 98%, 99% or 100% identical to the amino acid sequence of SEQ ID NO: 18 and a light chain variable region sequence that is at least about 95%, 96%, 97%, 98%, 99% or 100% identical to the amino acid sequence of SEQ ID NO: 19.

26. The method of claim 24, wherein the first antigen binding moiety of the T cell bispecific antibody is a crossover Fab molecule wherein the variable regions of the Fab light chain and the Fab heavy chain are exchanged, and
wherein the second and third antigen binding moiety of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

27. The method of claim 24, wherein the Fc domain of the T cell bispecific antibody comprises at least one of:
(i) a modification promoting the association of the first and the second subunit of the Fc domain, and
(ii) the Fc domain comprises one or more amino acid substitution that reduces binding to an Fc receptor and/or effector function.

28. The method of claim 18, wherein the T cell bispecific antibody is glofitamab.

29. The method of claim 3 or 7, wherein the disease is cancer.

30. The method of claim 29, wherein the cancer is (i) a CD20-expressing cancer or (ii) a B-cell cancer.

31. The method of claim 30, wherein the cancer is selected from the group consisting of Non-Hodgkin lymphoma (NHL), acute lymphocytic leukemia (ALL), chronic lymphocytic leukemia (CLL), diffuse large B-cell lymphoma (DLBCL), high grade B cell lymphoma (HGBCL), primary mediastinal large B-cell lymphoma (PMBCL), follicular lymphoma (FL), mantle-cell lymphoma (MCL) and marginal zone lymphoma (MZL).

32. The method of claim 11, wherein the inhibition is a complete inhibition, or a clinically meaningful and/or statistically significant inhibition.

33. The method of claim 13, wherein the cytokine is selected from the group consisting of IL-1β, IL-6 and IL-8.

34. The method of claim 15, wherein the administration of the NLRP3 inhibitor is intravenous.

35. The method of claim 17, wherein the administration of the T cell engaging agent is intravenous.

36. The method of claim 24, wherein the Fab molecule for each of the second and third antigen binding moiety is a conventional Fab molecule.

37. The method of claim 29, wherein the disease is a cancer expressing the target cell antigen of the T cell engaging agent.
